(19) United States
(12) Patent Application Publication
(10) Pub. No.: US 2018/0140532 A1
(43) Pub. Date: May 24, 2018

(54) COMPOSITIONS AND METHODS FOR RESTRUCTURING CHEMICALLY TREATED HAIR

(71) Applicant: L'OREAL, Paris (FR)

(72) Inventors: Jim Singer, South Orange, NJ (US); Andrea Elsen, Linden, NJ (US)

(21) Appl. No.: 15/356,967

(22) Filed: Nov. 21, 2016

(51) Int. Cl. A61K 8/85; A61Q 5/06; A61K 8/362; A61K 8/41

(52) U.S. Cl. CPC A61K 8/85; A61K 8/41; A61K 8/362; A61Q 5/06

(57) ABSTRACT

The present disclosure relates to hair care compositions such as hair restructuring compositions and composition for chemically treating hair comprising restructuring components. The disclosure also relates to methods for using the compositions to treat hair. The compositions and methods are unique because they dramatically improve the quality and durability of chemically treated hair. Typically, the compositions include at least: (a) one or more non-polymeric, mono-, di-, and/or tri-carboxylic acids; (b) one or more amines; and (c) one or more polymeric acid compounds and/or polymeric acid anhydride compounds.

### FIELD OF THE DISCLOSURE

The present disclosure relates to compositions and methods for treating hair, kits comprising the compositions, and methods for using the compositions.

### BACKGROUND

Many consumers desire to use cosmetic and care compositions that enhance the appearance of keratinous substrates such as hair, e.g., by changing the color, style, and/or shape of the hair, and/or by imparting various cosmetic properties to hair, such as shine and conditioning. Many of the known compositions and processes for enhancing the appearance of hair involve chemical treatments to the hair. The process of changing the color of hair, for example, can involve depositing an artificial color onto the hair which provides a different shade or color to the hair, and/or lifting the color of the hair, such as lightening the color of dark hair to lighter shades. The process of lifting the color of hair, also known as lightening (or bleaching), generally requires the use of oxidizing agents. Lightening of hair is typically evaluated by the variation in tone height before and after the application of a hair color-altering composition onto hair. This variation corresponds to the degree or level of lightening or lift. The notion of “tone” is based on the classification of the natural shades, one tone separating each shade from the shade immediately following or preceding it, which is well known to hairstyling professionals. The tone heights or levels can range from 1 (black) to 10 (light blond), one unit corresponding to one tone; thus, the higher the number, the lighter the shade or the greater the degree of lift.

In general, hair lightening or color lifting compositions and hair coloring or dyeing compositions possess an alkalinity such that these compositions have a pH value of above 7, typically being at pH 9 and above, and may generally require the presence of an alkalizing agent such as ammonia or an ammonia gas generating compound and/or an amine or ammonium-based compound in amounts sufficient to make such compositions alkaline. The alkalizing agent causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair.

Additionally, there are many techniques and compositions for styling or altering the shape of hair. For example, hair care products referred to as “hair relaxers” or “hair straighteners” can relax or straighten curly or kinky hair, including wavy hair. Straightening or relaxing the curls of very curly hair may increase the manageability and ease of styling of such hair. Compositions for permanent waving the hair will impart a curl or a wave to otherwise straight hair. Different types of compositions can be applied onto hair in order to change its shape and make it more manageable, such as alkaline and acidic compositions. Hair relaxers, straighteners, perms, and/or waves may either be applied in a hair salon by a professional or in the home by the individual consumer.

While dyeing or color lifting compositions can effectively alter the color of hair, and relaxing, straightening, perming, and waving compositions can effective alter the shape of the hair, these chemical treatments can damage the hair fibers and/or irritate the scalp. Thus, in order to reduce or avoid damage to hair, as well as to improve the cosmetic performance of the compositions, the use of new and additional components and novel combinations of ingredients are continuously sought.

However, the choice of components or combinations of ingredients could pose difficulties insofar as they cannot be detrimental to other cosmetic attributes such as ease and uniformity of application, rheology or viscosity properties and stability of the compositions, color deposit and target shade formation, and/or result into more disadvantages such as increased damage or a less healthy look to the hair. It would therefore be desirable to provide the consumer with compositions and methods that can chemically treat the hair while providing other cosmetic advantages such as shine, conditioning, fiber strength, and/or a healthy appearance to the hair, but avoiding or minimizing damage to the hair.

Further, both natural and sensitized or chemically treated hair can contain several kinds of negatively charged moieties, for example, carboxylates (resulting from the hydrolysis of amino acids and thioester bonds) and/or sulfonates (resulting from the oxidation of disulfide bonds). These negatively charged moieties can degrade the cosmetic properties of the hair. Moreover, when hair is chemically treated or damaged, the disulfide bonds in hair (disulfide linkages between two cysteine units) can be reduced or broken, resulting in the formation of thiol groups and/or cysteic acid.

Thus, one objective of the disclosure is to provide novel compositions that can provide advantageous effects such as strengthening of the hair fiber, protecting hair fibers from damage or further damage, enhanced properties such as softness, shine, conditioning, healthy appearance, while at the same time, providing desired effects such as coloring, lightening, straightening, relaxing, and/or shaping.

### SUMMARY OF THE DISCLOSURE

The present disclosure relates to compositions and methods for treating hair, in particular human hair of the head. The compositions and methods are unique because they dramatically improve the quality and durability of chemically treated hair. Damage during chemical treatment is repaired, minimized, and/or compensated for due to a unique combination of components in the compositions that restructure, strengthen, or protect the keratin fibers of the hair. Hair treated with the compositions is surprisingly strengthened and the hair's cosmetic properties (e.g., softness, smoothness, and discipline) are considerably improved.

The unique combination of components that function to restructure the hair can be combined in an individual restructuring composition and applied to the hair before, after, or during a chemical treatment; or the unique combination of components can be included with the composition that chemically treats the hair (e.g., in a composition that further includes one or more active agent for chemically treating hair).

In one aspect, the instant disclosure relates to restructuring compositions that include: (a) one or more non-polymeric, mono-, di-, and/or tri-carboxylic acids; (b) one or more amines; and (c) one or more polymeric acid compounds and/or polymeric acid anhydride compounds.

The hair restructuring compositions are applied to the hair in methods for restructuring the hair. Typically, the restructuring compositions are applied to the hair within about 24 hours before or after chemically treating the hair. The hair restructuring compositions may also be applied to the hair simultaneously with a chemical treatment or added directly to a chemical treatment composition and applied as part of the chemical treatment composition. Non-limiting examples of chemical treatments include lightening, coloring, perming, and straightening of the hair. The methods do not require the use of heat. Accordingly, the methods may be carried out at room temperature (about 20° C. to about 30° C.) or at a temperature of about 10° C. to about 50° C.

As mentioned above, the hair structuring compositions can be added and mixed with a chemical treatment composition immediately before application to hair. In some cases, however, components of the hair structuring compositions can already be part of a composition for chemically treating hair, i.e., the unique components of the hair restructuring compositions (“restructuring components”) can be formulated as part of a composition for chemically treating hair. This eliminates the step of separately applying a restructuring composition to the hair and/or eliminates the need for adding the hair restructuring composition to a chemical treatment composition before use. It also avoids the need for packing the restructuring composition separately from the chemical treatment composition.

The compositions for chemically treating hair typically include: (a) one or more non-polymeric, mono-, di-, and/or tri-carboxylic acids; (b) one or more amines; (c) one or more polymeric acid compounds and/or polymeric acid anhydride compounds; and (d) one or more active agents. The active agents are typical the compounds responsible, at least in part, for the chemically active nature of the compositions. Active agents include, for example, oxidizing agents, reducing agents, non-reducing agents for shaping hair, dyeing agents, and mixtures thereof. Accordingly, depending on the nature of the active agent(s), the composition for chemically treating the hair may be a hair lightening or bleaching composition, a hair coloring composition, a hair curling (perming) composition, a hair straightening composition, an anti-frizz composition, a hair smoothing composition, a hair conditioning composition, etc.

The compositions for chemically treating hair (the compositions already including the restructuring components) may be stand-alone compositions or may be used with or added to other compositions. For example, if the composition for chemically treating hair is a bleaching composition, it may be combined with a developer composition immediately before use. It is noted that either or both of the hair bleaching composition and the developer composition could include the hair restructuring components. Likewise, hair perming and hair straightening often requires more than one composition (e.g., relaxing compositions, activator compositions, neutralizer compositions, etc.). The restructuring components can be included in one or more (or all) of the various compositions.

The instant disclosure also relates to methods for chemically treating hair. The compositions of the instant disclosure are applied to the hair and allowed to remain on the hair for a period of time sufficient to achieve a desired result. Typically, a composition is allowed to remain on the hair for about 1 min. to about 45 min. and then the composition is rinsed from the hair. The methods do not require the use of heat. Accordingly, the methods may be carried out at room temperature (about 20° C. to about 30° C.) or at a temperature of about 10° C. to about 50° C.

The instant disclosure relates to kits, which include one or more compositions described herein. For instance, in some cases, the kits include: (i) a restructuring composition as described above; (ii) a composition comprising one or more active agents for chemically treating hair; and (iii) optionally, a composition comprising one or more hair conditioning agents; wherein each of the compositions is maintained separately in the kit. The instant disclosure also relates to kits in instances where the restructuring components are already included in one or more compositions for chemically treating hair. For example, these kits may include: (i) a composition for chemically treating hair, as described above; (ii) a composition comprising one or more second active agents that may be different than the one or more active agents in (i); and (iii) optionally, a composition comprising one or more hair conditioning agents.

Finally, as referenced previously, the instant disclosure relates to methods of using the compositions to treat hair or to restructure hair. These methods include applying one or more compositions of the instant disclosure to the hair. The one more composition may be temporarily applied to the hair (e.g., applied for a period of about 1 hour or less), or may be allowed to remain on the hair for an indefinite amount of time. When the hair restructuring components are included with a composition for chemically treating the hair, the composition for chemically treating the hair is typically applied to the hair for an amount of time sufficient to carry out the desired chemical treatment (e.g., less than about 1 hour). The compositions for chemically treating the hair are then usually rinsed from the hair after the desired amount of chemical treatment has been achieved.

### DETAILED DESCRIPTION OF THE DISCLOSURE

The instant disclosure relates to restructuring hair, in particular human hair of the head. In the context of the instant disclosure, the term “restructuring” relates to strengthening hair and minimizing and/or compensating for the damage caused to hair during chemical processing. Thus, a “restructuring composition” relates to a composition that strengthens hair by minimizing and/or compensating for the damage caused to the hair during chemical processing. Restructured hair fibers (hair treated with the restructuring compositions) exhibit desirable cosmetic properties such as smoothness, gloss, improved combability, and improved strength and elasticity.

The unique combination of components that function to restructure the hair can be combined in an individual restructuring composition and applied to the hair before, after, or during a chemical treatment; or the unique combination of components can be included as part of the a composition that chemically treats the hair.

Restructuring compositions according to the instant disclosure typically include:
(a) one or more non-polymeric, mono-, di-, and/or tri-carboxylic acids;
(b) one or more amines; and
(c) one or more one or more polymeric acid compounds and/or polymeric acid anhydride compounds.

Non-limiting examples of tri-carboxylic acids include ctric acid, isocitric acid, aconitric acid, propane-1,2,3-tricarboxylic acid, benzene-1,3,5-tricarboxylic acid, and mixtures thereof.

Non-limiting examples of di-carboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, and mixtures thereof.

Non-limiting examples of mono-carboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, and mixtures thereof.

In some cases, the restructuring composition comprises at least one or more di-carboxylic acids, for example, one or more di-carboxylic acids selected from the group consisting of maleic acid, malonic acid, and mixtures thereof. In some instances, the restructuring compositions include at least maleic acid.

The total amount of the one or more mono-, di-, and/or tri-carboxylic acids may vary but in some cases, the total amount is about 1 to about 50 wt. %, based on the total weight of the restructuring composition. In some cases, the total amount of the one or more mono-, di-, and/or tri-carboxylic acids is about 1 to about 40 wt. %, about 1 to about 30 wt. %, about 1 to about 25 wt. %, about 1 to about 20 wt. %, about 5 to about 50 wt. %, about 5 to about 40 wt. %, about 5 to about 30 wt. %, about 5 to about 25 wt. %, or about 5 to about 20 wt. %.

Non-limiting examples of the types of amines that may be used in the restructuring compositions are vast, but may include monoamines, diamines, polyamines, and mixtures thereof. The one or more amines may be primary, secondary, tertiary amines, and mixtures thereof. The monoamines and diamines may be alkyl amines, amidoamines, amino silicones, alkoxylated monoamines, and mixtures thereof. Non-limiting examples include am inosilicones, polyvinylamines, aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof. In some cases, the composition includes one or more monoamines selected from the group consisting of monoethanolamine, propanolamine, isopropanolamine, triethanolamine, butanolamine, isobutanolamine, methylethanolamine, butylethanolamine, and mixtures thereof.

Non-limiting examples of diamines include ethylenediamine (1,2-diaminoethane), 1,3-diaminopropane (propane-1,3-diamine), putrescine (butane-1,4-diamine), cadaverine (pentane-1,5-diamine), hexamethylenediamine (hexane-1,6-diamine), 1,2-diam inopropane, diphenylethylenediamine, diaminocyclohexane, xylylenediamine (o-xylylenediamine, m-xylylenediamine, and p-xylylenediamine), phenylenediamine (o-phenylenediamine, m-phenylenediamine, p-phenylenediamine), 2,5-diaminotoluene, dimethyl-4-phenylenediamine, N,N′-di-2-butyl-1,4-phenylenediamine, 4,4′-diaminobiphenyl, 1,8-diaminonaphthalene, and mixtures thereof.

Polyamines have more than 2 amino groups and may be a polymer comprising multiple amino groups including homopolymers, copolymers, and terpolymers. For instance, the polyamine may be an alkoxylated polyamine having at least two amino groups and at least one degree of alkoxylation. The alkoxylation is provided by an alkylene oxide group such as, for example, ethylene oxide and/or propylene oxide. In some cases, the compositions do not include polymers having dimethylamino moieties, i.e., the compositions are free of essentially free of polyamines that are polymers having dimethylamino moieties.

In some instances, the compositions of the instant disclosure are free or essentially free of VP/DMAPA acrylates copolymer. VP/DMAPA acrylates copolymer is a copolymer of vinylpyrrolidone anddimethylaminopropylacrylamide or methacrylamide. In some cases, when the compositions of the instant disclosure include an oxidizing agent, the compositions are free or essentially free of VP/DMAPA acrylates copolymer. In some cases, when the compositions of the instant disclosure include hydrogen peroxide, the compositions are free or essentially free of VP/DMAPA acrylates copolymer. Furthermore, individual polyamines that may be include or excluded from the instant compositions include include polyethyleneimines, polyvinylamines, aminated polysaccharides, amine substituted polyalkylene glycols, amine substituted polyacrylate crosspolymers, amine substituted polyacrylates, amine substituted polymethacrylates, amine substituted polyesters, polyamino acids, polyalkylamines, diethylene triamines, triethylenetetramines, and mixtures thereof. Polyimide-1 may also optionally be included or excluded, or present in an amount less than about 5 wt. %, about 4 wt. %, about 3 wt. %, about 2 wt. %, about 1 wt. %, about 0.5 wt. %, about 0.1 wt. %, about 0.05 wt. % or about 0.01 wt. %.

The total amount of the one or more amines may vary, but in some cases, the total amount of the one or more amines is about 0.1 to about 50 wt. %, based on the total weight of the restructuring composition. In some cases, the total amount of the one or more amines is about 0.1 to about 50 wt. %, about 0.1 to about 30 wt. %, about 0.1 to about 25 wt. %, about 0.1 to about 20 wt. %, about 0.1 to about 15 wt. %, about 0.1 to about 10 wt. %, about 0.5 to about 35 wt. %, about 0.5 to about 30 wt. %, about 0.5 to about 25 wt. %, about 0.5 to about 20 wt. %, about 0.5 to about 15 wt. %, about 0.5 to about 10 wt. %, about 1 to about 35 wt. %, about 1 to about 30 wt. %, about 1 to about 25 wt. %, about 1 to about 20 wt. %, about 1 to about 15 wt. %, about 1 to about 10 wt. %, or about 3 to about 8 wt. %.

The one or more polymeric acid compounds and/or polymeric acid anhydride compounds has recurring units. The recurring units may be derived from one more of the following acids (or anhydrides, salts, or derivatives of the acids):
(i) styrene sulfonic acid,
(ii) crotonic acid,
(iii) acrylic or methacrylic acid,
(iv) ethylene-a,β-dicarboxylic acid, and/or
(v) allyloxyacetic acid, methallyloxyacetic acid, 3-allyloxypropionic acid, allylthioacetic acid, allylaminoacetic acid, vinylacetic acid, vinyloxyacetic acid, crotyloxyacetic acid, 3-butenoic acid, 4-pentenoic acid, 10-undecenoic acid, allylmalonic acid, maleamic acid, itaconamic acid or N-monohydroxyalkyl- or N-dihydroxy-alkyl-maleamic or -itaconamic acids.

In some cases, the one or more polymeric acid compounds and/or polymeric acid anhydride compounds are ethylene-α,β-dicarboxylic acids, anhydrides, salts, or derivatives thereof. Suitable α,β-unsaturated carboxylic acids for copolymerization with ethylene are α,β-unsaturated aliphatic mono- or di-carboxylic acids having 3 to 8 or 3 to 5 carbon atoms. Non-limiting examples include acrylic acid, methacrylic acid, α-ethylmethacrylic acid, maleic acid, fumaric acid, and itaconic acid anhydrides, salts, or derivatives thereof. In some instances in particular, maleic acid, anhydrides, salts, or derivatives thereof are appropriate. Accordingly, the instant disclosure relates to compositions that include: (a) one or more polymers containing recurring units derived from maleic acid and/or maleic anhydride; and (b) one or more active agents.

Non-limiting examples of polymeric acid compounds and/or polymeric acid anhydride compounds include maleic anhydride copolymers such as poly(styrene-alt-maleic anhydride), poly(methyl vinyl ether-alt-maleic anhydride), poly(ethylene-g-maleic anhydride), poly(isobutylene-alt-maleic anhydride), polyisoprene-g-maleic anhydride, poly(maleic anhydride-alt-1-octadecene), poly(ethylene-co-ethyl acrylate-co-maleic anhydride), polyethylene-graft-maleic anhydride, and mixtures thereof.

Further non-limiting examples of polymeric acid compounds and/or polymeric acid anhydride compounds include maleic acid copolymers such as butyl ester of ethylene/MA copolymer, butyl ester of PVM/MA copolymer, calcium/sodium salt of PVM/MA copolymer, ethyl ester of PVM/MA copolymer, ethylene/MA copolymer, isobutylene/MA copolymer, isopropyl ester of PVM/MA copolymer, octadecene/MA copolymer, polyethylene/isopropyl maleate/MA copolymer, polyvinyl methyl ether (PVM)/MA copolymer, PVM/MA decadiene crosspolymer, sodium C4-C12 olein/maleic acid copolymer, sodium isooctylene/MA copolymer, sodium MA/diisobutylene copolymer, sodium PVM/MA/decadiene crosspolymer, stearylvinyl ether/MA copolymer, styrene/MA copolymer, and mixtures thereof.

The total amount of the polymeric acid compounds and/or polymeric acid anhydride compounds may vary. For example, in some cases the total amount is about 0.01 to about 25 wt. %, based on the total weight of the composition. However, the total amount of the one or more polymeric acid compounds and/or polymeric acid anhydride compounds may be about 0.01 to about 20 wt. %, about 0.01 to about 15 wt. %, about 0.01 to about 10 wt. %, about 0.01 to about 8 wt. %, 0.01 to about 5 wt. %, about 0.01 to about 4 wt. %, about 0.01 to about 3 wt. %, about 0.1 to about 10 wt. %, about 0.1 to about 8 wt. %, about 01 to about 5 wt. %, about 0.1 to about 4 wt. %, or about 0.1 to about 3 wt. %.

The components of the hair structuring compositions may already be part of a composition for chemically treating hair. The unique components of the hair restructuring compositions are formulated as part of a composition for chemically treating hair. Thus, the instant disclosure relates to compositions for chemically treating hair that typically include:
(a) one or more non-polymeric, mono-, di-, and/or tri-carboxylic acids;
(b) one or more amines; and
(c) one or more polymeric acid compounds and/or polymeric acid anhydride compounds; and
(d) one or more active agents.

An “active agent,” in the context of the instant disclosure, relates to a compound, molecule, or combination of compounds/molecules that chemically changes hair. For example, an active agent may reduce disulfide bonds, reestablish or form disulfide bonds, remove melanin from the hair, covalently bond to the hair, etc. Non-limiting examples of active agents include oxidizing agents, reducing agents, non-reducing agents for shaping hair, dyeing agents, and mixtures thereof. Based on the type of active agent, the composition for chemically treating hair may be a hair lightening or bleaching composition, hair coloring composition, a hair perming or straightening composition, or a mixture thereof.

Hair lightening compositions typically include one or more oxidizing agents. Non-limiting examples of oxidizing agents include peroxides, persulfates, perborates, percarbonates, and mixtures thereof. In some cases, the hair lightening composition includes one or more persulfates, such as those selected from the group consisting of sodium persulfate, potassium persulfate, ammonium persulfate, and mixtures thereof. In some cases, the hair lightening compositions include peroxide, such as hydrogen peroxide.

Hair perming or straightening compositions typically include one or more reducing agents. Non-limiting examples of reducing agents include cysteine or a derivative of cysteine, cysteamine or a derivative of cysteamine, thiolactic acid or an ester of thiolactic acid, thioglycolic acid or an ester of thioglycolic acid, thioglycerol, and mixtures thereof. In some cases, the reducing agent is a glyceryl or glycol monothioglycolate, diammonium dithiodiglycolate, ammonium thioglycolate, or a mixture thereof.

Hair straightening or relaxing compositions may include one or more non-reducing agents for shaping hair. Non-reducing agents for shaping hair may be one or more hydroxide compounds, non-hydroxide compounds, or mixtures thereof. For instance, the hydroxide compounds may be alkali metal hydroxides, alkaline-earth metal hydroxides, transition metal hydroxides, and mixtures thereof. Non-limiting examples include of hydroxide compounds include sodium hydroxide, potassium hydroxide, lithium hydroxide, calcium hydroxide, magnesium hydroxide, barium hydroxide, strontium hydroxide, manganese hydroxide, zinc hydroxide, guanidine hydroxide, and mixtures thereof.

The non-hydroxide compounds may include one or more ethyleneamines, alkanolamines, amino acids, or mixtures thereof. Non-limiting examples of non-hydroxide compounds include ethylenediamine, monoethanolamine, diethanolamine, propanolamine, isopropanolamine, triethanolamine, butanolamine, isobutanolamine, methylethanolamine, butylethanolamine, arginine, lysine, and mixtures thereof.

In some cases, when the compositions include hydrogen peroxide as an active agent, the composition further comprises a second oxidizing agent other than hydrogen peroxide and/or the compositions includes an oxidative dye precursor. For example, in some cases the second oxidizing agent is a persulfate. Non-limiting examples of persulfates include potassium persulfate, sodium persulfate, ammonium persulfate, and mixtures thereof.

Hair coloring or dyeing compositions typically include one or more colorants or dyeing agents. Non-limiting examples of colorants or dyeing agents include direct dyes, oxidative dyes, direct action dyes, natural dyes, metallic dyes, reactive dyes, and mixtures thereof.

The compositions for chemically treating hair can include varying amounts of components depending on the purpose of the composition and depending on whether the composition will be combined with additional compositions. Typically, when the restructuring components are already included in a composition with the one or more active agents, the total amount of each of the restructuring agents will be less than for a restructuring composition that includes primarily the restructuring agents. The total amount of each of the restructuring components will be reduced relative to the total weight of the composition due to the addition of the one or more active agents and the additional components included in the composition for chemically treating hair.

With respect to the total amount of the non-polymeric, mono-, di-, and/or tri-carboxylic acids, the composition may include about 0.01 to about 25 wt. %, about 0.01 to about 20 wt. %, 0.01 to about 15 wt. %, about 0.01 to about 10 wt. %, about 0.01 to about 5 wt. %, about 0.01 to about 4 wt. %, about 0.01 to about 3 wt. %, about 0.1 to about 20 wt. %, about 0.01 to about 15 wt. %, about 0.1 to about 10 wt. %, about 0.1 to about 5 wt. %, about 0.1 to about 4 wt. %, about 0.1 to about 3 wt. %, about 0.5 to about 4 wt. %, about 0.5 to about 3 wt. %, or about 1 to about 3 wt. %.

With respect to the total amount of the one or more amines, the composition may include about 0.01 to about 25 wt. %, about 0.01 to about 20 wt. %, about 0.01 to about 15 wt. %, 0.01 to about 10 wt. %, about 0.01 to about 8 wt. %, about 0.01 to about 4 wt. %, 0.01 to about 3 wt. %, about 0.01 to about 2 wt. %, about 0.05 to about 20 wt. %, about 0.05 to about 15 wt. %, about 0.05 to about 10 wt. %, about 0.05 to about 8 wt. %, about 0.05 to about 4 wt. %, about 0.05 to about 3 wt. %, 0.1 to about 15 wt. %, 0.01 to about 10 wt. %, 0.1 to about 5 wt%, about 0.01 to about 2 wt. %, or about 0.1 to about 2 wt. %.

With respect to the total amount of the one or more one or more polymeric acid compounds and/or polymeric acid anhydride compounds, the compositions may include about 0.01 to about 5 wt. %, about 0.01 to about 3 wt. %, about 0.01 to about 2 wt. %, about 0.01 to about 1 wt. %, about 0.01 to about 5 wt. %, about 0.01 to about 3 wt. %, about 0.01 to about 2 wt. %, about 0.1 to about 5 wt. %, about 0.1 to about 4 wt. %, about 0.1 to about 3 wt. %, about 0.1 to about 2 wt. %, or about 0.1 to about 1 wt. %.

With respect to the total amount of the one or more active agents, the composition may include about 0.01 to about 85 wt. %, about 0.01 to about 50 wt. %, about 0.01 to about 25 wt. %, about 0.01 to about 20 wt. %, about 0.01 to about 10 wt. %, about 0.01 to about 5 wt. %, about 1 to about 85 wt. %, about 1 to about 70 wt. %, about 1 to about 60 wt. %, about 1 to about 50 wt. %, about 1 to about 25 wt. %, or about 1 to about 10 wt. %, about 25 to about 95 wt. %, about 40 wt. % to about 95 wt. %, or about 50 wt. % to about 80 wt. %.

The instant disclosure further relates to methods for treating and/or restructuring hair using the restructuring compositions and the compositions for chemically treating hair. For instance, the instant disclosure relates to methods for restructuring hair, the methods comprising applying a hair restructuring composition to the hair. The hair restructuring composition may be temporarily applied to the hair for a sufficient amount of time to restructure the hair and then removed, for example, by rinsing. Alternatively, the restructuring composition may be a “leave-in” composition that does not require removal from the hair.

The methods are unique because they do not require heat to attain the dramatic improvements in cosmetic and/or sensorial properties (e.g., softness, smoothness, discipline, conditioning, etc.). Thus, the methods are typically carried out at room temperature (about 20° C. to about 30° C.). In particular, methods can be performed without allowing the hair and the compositions applied to the hair to reach a temperate above about 50 ° C. or about 60° C. The methods can be performed without applying heat during treatment or after treatment. Accordingly, the methods may be carried out at a temperature of about 10° C. to about 60° C., about 15° C. to about 50° C., about 15° C. to about 40° C., or about 15° C. to about 30° C.

The restructuring composition may be applied to the hair within about 1 hour, about 45 min., about 30 min., about 20 min., about 15 min., about 10 min., or about 5 min. of chemically treating the hair (before or after the chemical treatment). For example, the restructuring composition may be applied to the hair followed by application of a chemical treatment composition. Alternatively, a chemical treatment composition may be applied to the hair followed by application of a restructuring composition. In some cases, a restructuring composition is applied both before and after a chemical treatment composition is applied to the hair. In some cases, when applying a restructuring composition to the hair after chemical treatment, the chemical treatment composition is first rinsed from the hair before application of the restructuring composition. The hair restructuring composition can be applied to the hair and allowed to dry on the hair, naturally or with the use of heat and/or air (blow dry). Without rinsing the restructuring composition from the hair, a chemical treatment composition can subsequently be applied to the hair. In some cases, a restructuring composition is applied to the hair within about 1 hour, about 45 min., about 30 min., about 20 min., about 15 min., about 10 min., or about 5 min. before application of a chemical treatment composition. Likewise, in some cases, a restructuring composition is applied to the hair within about 1 hour, about 45 min., about 30 min., about 20 min., about 15 min., about 10 min., or about 5 min. after application of a chemical treatment composition. In some cases, the restructuring composition is applied simultaneously with a hair treatment composition or is added directly to the hair treatment composition before application to the hair.

If the hair treatment composition is for lightening hair, a method for lightening hair can include: (i) mixing a hair restructuring composition with a bleaching composition, and optionally with a composition comprising one or more hair conditioning agents and/or one or more direct dyes; (ii) applying the mixture onto hair and allowing the mixture to remain on the hair for about 1 to about 45 minutes; and (iii) rinsing the mixture from hair. If the hair treatment composition is for perming or straightening the hair, a method for perming and/or straightening the hair may include: (i) mixing a restructuring composition with a reducing compositions and/or with a neutralizing composition; and (ii) applying the reducing composition and/or the neutralizing composition to the hair for about 1 to about 45 minutes. Usually, the reducing composition and the neutralizing composition are applied sequentially.

The instant disclosure also relates to methods for chemically treating using chemical compositions already formulated to include the restructuring agents of the restructuring compositions. Typically, the compositions for chemically treating hair are applied to the hair for a sufficient amount of time to attain a desired result, and then rinsed from the hair. The compositions for chemically treating hair may be applied to the hair for about 1 hour, about 45 min, about 30 min, about 20 min, about 15 min, about 10 min, or about 5 min., and then rinsed from the hair. For example, if the composition for chemically treating hair is a hair lightening composition, a method for lightening the hair may include: (i) mixing a hair lightening composition with a developer composition comprising a peroxide, and optionally with a composition comprising one or more hair conditioning agents and/or one or more direct dyes; (ii) applying the mixture onto hair and allowing the mixture to remain on the hair for about 1 to about 45 minutes; and (iii) rinsing the mixture from hair. In some cases, however, the restructuring components may be formulated with the developer composition comprising a peroxide, such that the developer composition instead of with the hair lightening composition. In some cases, the restructuring components may be included in both the hair lightening composition and the developer composition.

The compositions described in the instant disclosure may be included in kits for treating hair. Kits typically include more than one composition and therefore more than one container (or more than one compartment in a given container) to ensure that various compositions do not come into contact with each other until desired. For example, the kits may include a restructuring composition, as described previously, or may include a composition for chemically treating hair (compositions already formulated to include the restructuring agents of the restructuring compositions). The kits may further include a developer bottle, gloves, shampoo, conditioner, and/or an odor eliminator. Instructions for use of the kit are also typically provided.

In some cases, the kits include: (i) a restructuring composition; (ii) a composition comprising one or more active agents for chemically treating hair; and (iii) optionally, a composition comprising one or more hair conditioning agents. In some cases, the kit further includes a second composition comprising one or more active agents (typically active agents that are different than in the composition of (ii)). For example, the composition of (ii) may include, as an active agent, one or more oxidizing agents selected from the group consisting of persulfates, perborates percarbonates, alkali metal bromates, ferricyanides, peroxygenated salts, and mixtures thereof. The second composition may be, for example, a developer composition that includes peroxide. Likewise, the composition of (ii) may include, as an active agent, one or more reducing agents for perming or straightening the hair and the second composition may be a neutralizing composition wherein the one or more active agents are reducing agents.

In instances where the restructuring components of the restructuring compositions are already formulated into a composition for chemically treating hair, the kits will typically include: (i) a composition for chemically treating hair; and (ii) optionally, a composition comprising one or more hair conditioning agents. Such kits may also include a composition comprising one or more second active agents (typically different than the active agents already in (i)). For example, if the composition for chemically treating hair is a hair lightening composition, the kit may include a developer composition. The restructuring agents may be included in either the hair lightening composition or the developer composition, or both. If the composition is for perming or straightening hair, the restructuring agents may be included in the reducing composition or in the neutralizing composition, or both.

Preservatives, conditioning agents including cationic conditioning agents, colorants or dyes, thickeners, surfactants including ionic surfactants, nonionic surfactants, amphoteric surfactants and/or zwitterionic surfactants, stabilizers, pH modifiers, buffers, etc., may also optionally be included (or excluded) from the compositions.

More exhaustive but non-limiting lists of components that are useful in the compositions of the instant disclosure are presented below.

### Mono-, Di, and Tri-Carboxylic Acids

The mono-, di-, and/or tri-carboxylic acids may be chosen especially from linear, branched and/or cyclic, saturated or unsaturated, or even aromatic, polycarboxylic acids, containing 2 to 50 or 2 to 40 carbon atoms, in particular 3 to 36, 3 to 18, or 4 to 12 carbon atoms, or even 5 to 10 carbon atoms; the acid comprising one, two, or three carboxylic groups COOH; and possibly comprising 1 to 10 or 1 to 6 identical or different heteroatoms, chosen from O, N and S; and/or possibly comprising at least one perfluoro radical chosen from —CF₂—(divalent) or —CF₃.

In some cases, the mono-, di-, and/or tri-carboxylic acids are saturated, linear and aliphatic and contain 2 to 36 carbon atoms or 3 to 18 carbon atoms or even 4 to 12 carbon atoms; or alternatively are aromatic and contain 8 to 12 carbon atoms.

The cyclic anhydride of a polycarboxylic acid may correspond to one of the following formulae:

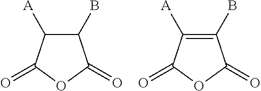

in which the groups A and B are, independently of each other: a hydrogen atom, a saturated or unsaturated, linear, branched and/or cyclic aliphatic, or alternatively aromatic, carbon-based radical; containing 1 to 16 carbon atoms, 2 to 10 carbon atoms or even 4 to 8 carbon atoms, especially methyl or ethyl, or alternatively A and B taken together form a saturated or unsaturated, or even aromatic, ring comprising in total 5 to 14, especially 5 to 10 or even 6 to 7 carbon atoms. In some cases, A and B represent a hydrogen atom or together form an aromatic ring containing in total 6 to 10 carbon atoms.

Among the mono-, di-, and/or tri-carboxylic acids or anhydrides thereof that may be used, mention may be made, alone or as a mixture, of: dicarboxylic acids such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid and fatty acid dimers (especially of C₃₆); tricarboxylic acids such as cyclohexanetricarboxylic acid, trimellitic acid, 1,2,3-benzenetricarboxylic acid and 1,3,5-benzenetricarboxylic acid, tetracarboxylic acids such as butanetetracarboxylic acid and pyromellitic acid, cyclic anhydrides of these acids and especially phthalic anhydride, trimellitic anhydride, maleic anhydride and succinic anhydride.

Mention may also be made of mono-, di-, and/or tri-carboxylic acids chosen, alone or as a mixture, from:

(i) mono-, di-, and/or tri-carboxylic acids containing a saturated or unsaturated, linear or branched chain comprising at least one heteroatom chosen from O, N and/or S, especially 1 to 10 identical or different heteroatoms, and/or comprising at least one perfluoro radical —CF₂₋ or —CF₃ and moreover containing 1, 2, or 3 carboxylic groups COOH; and/or a cyclic anhydride of such a polycarboxylic acid; and/or

(ii) saturated or unsaturated, or even aromatic, heterocyclic mono-, di-, and/or tri-carboxylic acids comprising at least one heteroatom chosen from O, N and/or S, especially 1 to 10, or even 1 to 4, identical or different heteroatoms, and 1, 2, or 3 carboxylic groups COOH; and/or a cyclic anhydride of such a polycarboxylic acid; and/or

(iii) sugar-based mono-, di-, and/or tri-carboxylic acids, which may be obtained especially by oxidation of an aldose, and comprising 1, 2, or 3 carboxylic groups COOH; and/or a cyclic anhydride of such a polycarboxylic acid; and/or

(iv) itaconic anhydride;

(v) mono-, di-, and/or tri-carboxylic acids (including heterocyclic) amino acids, i.e. polycarboxylic acids containing a saturated or unsaturated, linear, branched and/or cyclic chain, optionally comprising at least one heteroatom chosen from O, N and/or S, especially 1 to 10 identical or different heteroatoms, and/or optionally comprising at least one perfluoro radical —CF₂₋ or —CF₃; and also comprising at least one primary, secondary or tertiary amine function (especially NR¹R² with R¹ and R², independently of each other, chosen from H and C₁-C₁₂ alkyl), especially 1 to 3 identical or different amine functions, and moreover containing 1, 2, or 3 carboxylic acid groups COOH; and/or a cyclic anhydride of such a polycarboxylic acid.

Mention may be made most particularly, alone or as a mixture, of the following di-carboxylic acids: (i) 2,2′-[1,5-pentanediylbis(thio)]bis-acetic acid 6,6′-[(1,2-dioxo-1,2-ethanediyl)diimino]bis-hexanoic acid 2,2′-sulfinylbis-acetic acid 4,13-dioxo-3,5,12,14-tetraazahexadecanedioic acid poly(ethylene glycol)disuccinate, especially of mass 250-600 poly(ethylene glycol)bis(carboxymethyl) ether, especially of mass 250-600 poly[oxy(1,2-dicarboxy-1,2-ethanediyl)], especially of DP<10 8-[(carboxymethyl)amino]-8-oxooctanoic acid 2,2′-[methylenebis(sulfonyl)]bis-acetic acid 4,4′-(1,6-hexanediyldiimino)bis[4-oxobutanoic acid] 4,9-dioxo-3,5,8,10-tetraazadodecanedioic acid 4-[(1-carboxyethyl)amino]-4-oxobutanoic acid 6-[(3-carboxy-1-oxopropyl)amino]hexanoic acid N,N′-(1,6-dioxo-1,6-hexanediyl)bis-glycine N,N′-(1,6-dioxo-1,6-hexanediyl)bis-phenylalanine N,N′-(1,3-dioxo-1,3-propanediyl)bis-glycine 4,4′-[(1,4-dioxo-1,4-butanediyl)diimino]bis-butanoic acid 4,4′-[(1,6-dioxo-1,6-hexanediyl)diimino]bis-butanoic acid 6,6′-[1,6-hexanediylbis(iminocarbonylimino)]bis-hexanoic acid N-benzoyl-S-(carboxymethyl)cysteine N,N′-(2,2,3,3-tetrafluoro-1,4-dioxo-1,4-butanediyl)bis-glycine N,N′-(2,2,3,3-tetrafluoro-1,4-dioxo-1,4-butanediyl)bis-alanine 4,4′-[(2,2,3,3-tetrafluoro-1,4-dioxo-1,4-butanoic acid N,N′-(1,5-dioxo-1,5-pentanediyl)bis-glycine N,N′-(1,9-dioxo-1,9-nonanediyl)bis-glycine N,N′-(1,10-dioxo-1,10-decanediyl)bis[N-methyl]glycine bis(3-carboxypropyl)ester of propanedioic acid 7,16-dioxo-6,8,15,17-tetraazadocosanedioic acid N-benzoyl-N-(2-carboxyethyl)glycine [2-[(2-carboxymethyl)amino]-2-oxoethyl]benzenepropanoic acid [2-[(2-carboxyethyl)amino]-2-oxoethyl]benzenepropanoic acid (ii) 4,7,9,12-tetraoxapentadecanedioic acid 2,3-pyridinedicarboxylic acid 4-pyranone-2,6-dicarboxylic acid 2,5-pyrazinedicarboxylic acid 2,5-pyridinedicarboxylic acid 2,3-benzofurandicarboxylic acid 7-oxabicyclo[2.2.1]heptane-2,3-dicarboxylic acid 3,4-pyridinedicarboxylic acid 2,4-pyridinedicarboxylic acid 3,5-pyridinedicarboxylic acid 2,6-pyridinedicarboxylic acid 1H-imidazole-4,5-dicarboxylic acid 2,3-quinolinedicarboxylic acid 6,6,7,7-tetrafluoro-3-oxabicyclo[3.2.0]heptane-2,4-dicarboxylic acid 2,6-pyrazinedicarboxylic acid 2,6-dimethyl-3,5-pyridinedicarboxylic acid 1-phenyl-1H-pyrazole-3,4-dicarboxylic acid 2,5-furandicarboxylic acid 3,4-furandicarboxylic acid 1,2,5-thiadiazole-3,4-dicarboxylic acid 1,4-dihydro-1,2,4,5-tetrazine-3,6-dicarboxylic acid 2,3-furandicarboxylic acid 3,4-thiophenedicarboxylic acid 1H-1,2,3-triazole-4,5-dicarboxylic acid 2-methylimidazole-4,5-dicarboxylic acid 2,4-quinolinedicarboxylic acid naphtho[2,1-b]furan-1,2-dicarboxylic acid 3,4-quinolinedicarboxylic acid 7-oxabicyclo[2.2.1]hept-5-ene-2,3-dicarboxylic acid 2,3-quinoxalinedicarboxylic acid 1,4-piperazinedicarboxylic acid 2,5-dimethyl-3,4-furandicarboxylic acid tetrahydro-2,5-thiophenedicarboxylic acid 4-phenyl-3,5-pyridinedicarboxylic acid thieno[3,2-b]thiophene-2,5-dicarboxylic acid 3-methyl-2,4-thiophenedicarboxylic acid naphthostyril-5,6-dicarboxylic acid 3-phenyl-2,4-quinolinedicarboxylic acid 3,4-dimethyl-2,5-dicarboxythiophene 3,4-diphenyl-2,5-thiophenedicarboxylic acid 2,5-diphenyl-3,4-furandicarboxylic acid 7-oxo-7H-benzimidazo[2,1-a]benz[de]isoquinoline-3,4-dicarboxylic acid 2,3-dihydro-1,3-dioxo-1H-benz[de]isoquinoline-6,7-dicarboxylic acid 3,4-bis(phenylmethoxy)-2,5-furandicarboxylic acid 4,4′-bibenzoic acid-2,2′-sulfone 2,7-diphenyl-m-anthrazoline-4,5-dicarboxylic acid 2,4-pyrimidinedicarboxylic acid 2-phenyl-4,5-thiazoledicarboxylic acid 6-phenyl-2,3-pyridinedicarboxylic acid 5,6-dimethyl-2,3-pyrazinedicarboxylic acid 3,7-dibenzothiophenedicarboxylic acid 9-oxo-9H-xanthene-1,7-dicarboxylic acid 241,1-dimethylethyl)-H-imidazole-4,5-dicarboxylic acid 6,7-quinolinedicarboxylic acid 6-methyl-2,3-pyridinedicarboxylic acid 4,5-pyrimidinedicarboxylic acid 2-methyl-3,4-furandicarboxylic acid 1,2-indolizinedicarboxylic acid 2,8-dibenzothiophenedicarboxylic acid 3,6-pyridazinedicarboxylic acid 1,10-phenanthroline-2,9-dicarboxylic acid 1,4,5,6-tetrahydro-5,6-dioxo-2,3-pyrazinedicarboxylic acid 3,4-dimethoxy-2,5-furandicarboxylic acid 2-ethyl-4,5-imidazoledicarboxylic acid 2-propyl-1H-imidazole-4,5-dicarboxylic acid 4-phenyl-2,5-pyridinedicarboxylic acid 4,5-pyridazinedicarboxylic acid 1,4,5,8-tetrahydro-1,4:5,8-diepoxynaphthalene-4a,8a-dicarboxylic acid 5,5-dioxide-2,8-dibenzothiophenedicarboxylic acid pyrazolo[1,5-a]pyridine-2,3-dicarboxylic acid 2,3-dihydro-1H-pyrrolizine-1,7-dicarboxylic acid 6-methyl-2,4,5-pyridinetricarboxylic acid pyrrolo[2,1,5-cd]indolizine-5,6-dicarboxylic acid 3,4-bis(2,2,3,3,4,4,4-heptafluorobutyl)-1H-pyrrole-2,5-dicarboxylic acid 6,7,9,10,17,18,20,21-octahydrodibenzo[b,k]-[1,4,7,10,13,16]hexaoxacyclooc- tadecin-2,14-dicarboxylic acid 6,7,9,10,17,18,20,21-octahydrodibenzo[b,k]-[1 ,4,7,10,13,16]hexaoxacyclooc- tadecin-2,13-dicarboxylic acid 2-methyl-3,4-quinolinedicarboxylic acid 4,7-quinolinedicarboxylic acid 3,5-isoxazoledicarboxylic acid 2-(trifluoromethyl)-3,4-furandicarboxylic acid 5-(trifluoromethyl)-2,4-furandicarboxylic acid 6-methyl-2,4-quinolinedicarboxylic acid 5-oxo-1,2-pyrrolidinedicarboxylic acid 5-ethyl-2,3-pyridinedicarboxylic acid 1,2-dihydro-2-oxo-3,4-quinolinedicarboxylic acid 4,6-phenoxathiindicarboxylic acid 10,10-dioxide 1,9-phenoxathiindicarboxylic acid 3,4-dihydro-2H-1,4-thiazine-3,5-dicarboxylic acid 2,7-di(tert-butyl)-9,9-dimethyl-4,5-xanthenedicarboxylic acid 6-methyl-2,3-quinoxalinedicarboxylic acid 3,7-quinolinedicarboxylic acid 2,5-quinolinedicarboxylic acid 2-methyl-6-phenyl-3,4-pyridinedicarboxylic acid 3,4-dimethylthieno[2,3-b]thiophene-2,5-dicarboxylic acid 3,4-dimethoxythiophene-2,5-dicarboxylic acid 5-methyl-3,4-isoxazoledicarboxylic acid 2,6-bis(aminocarbonyl)-3,5-pyridinedicarboxylic acid 3,5-bis(aminocarbonyl)-2,6-pyrazinedicarboxylic acid 2,3-pyridinedicarboxylic acid 6-(1,1-dimethylethyl)-2-ethyl-3,4-pyridinedicarboxylic acid 3-methyl-5-phenyl-2,4-thiophenedicarboxylic acid 1,2-dihydro-2-oxo-6-phenyl-3,5-pyridinedicarboxylic acid 8-methyl-2,4-quinolinedicarboxylic acid 4-ethyl-2,6-dimethyl-3,5-pyridinedicarboxylic acid 5-(phenoxymethyl)-2,4-furandicarboxylic acid 5-(acetylamino)-3-methyl-2,4-thiophenedicarboxylic acid 2-(4-heptylphenyl)-4,8-quinolinedicarboxylic acid 2,8-bis(4-heptylphenyl)pyrido[3,2-g]quinoline-4,6-dicarboxylic acid 1,2,3,4,6,7,8,9-octahydro-2,8-dioxopyrido[3,2]-quinoline-3,7-dicarboxylic acid 2,8-dimethylpyrido[3,2-g]quinoline-3,7-dicarboxylic acid 5,6-quinolinedicarboxylic acid 6-ethyl-2-methylcinchomeronic acid 2-methyl-6-propylcinchomeronic acid 6-isopropyl-2-methylcinchomeronic acid 6-tert-butyl-2-methylcinchomeronic acid 1,4-dimethyl-7-oxabicyclo[2.2.1]heptane-2,3-dicarboxylic acid 1,2-dihydro-2-oxo-3,8-quinolinedicarboxylic acid 1,2-dihydro-2-oxo-3,6-quinolinedicarboxylic acid 1,2-dihydro-2-oxo-3,7-quinolinedicarboxylic acid 3,7-dimethyl-2,8-diphenylpyrido[3,2-g]quinoline-4,6-dicarboxylic acid 8-methyl-2,3-quinolinedicarboxylic acid 3-[[(1 ,1-dimethylethyl)amino]sulfonyl]-2,5-thiophenedicarboxylic acid 4-(acetylamino)-2,3-thiophenedicarboxylic acid 2,5-pyridinedicarboxylic acid 2,6-pyridinedicarboxylic acid 2,4-thiophenedicarboxylic acid 2,5-thiophenedicarboxylic acid 1,4-pyran-2,6-dicarboxylic acid (iii) ribaric acid glucaric acid xylaric acid arabinaric acid mannaric acid idaric acid altraric acid L-glucaric acid L-arabinaric acid allaric acid galactaric acid meso-tartaric acid D-glucaric acid L-idaric acid hexaric acid 2,3-dihydroxybutanedioic acid D-tartaric acid D,L-tartaric acid D-glucaric acid tartaric acid tetrahydroxysuccinic acid 2-carboxy-2,3-dideoxy-D-manno-2-octulopyranosonic acid methyl-3-deoxy-D-arabino-2-heptulopyranosaric acid D-lyxo-2-heptulopyranosaric acid 2,6-anhydro-L-glycero-L-galactoheptaric acid (iv) 1,4,5,8-naphthalenetetracarboxylic acid 1,4-monoanhydride itaconic anhydride (v) 1,4-dihydro-4-oxo-2,6-pyridinedicarboxylic acid 2,6-piperidinedicarboxylic acid 1H-pyrrole-3,4-dicarboxylic acid 4-amino-2,6-dicarboxylic acid 1-methyl-1H-pyrazole-3,4-dicarboxylic acid 2,3-piperidinedicarboxylic acid 1-methyl-1H-imidazole-4,5-dicarboxylic acid 2,4-thiazolidinedicarboxylic acid 1-(phenylmethyl)-1H-imidazole-4,5-dicarboxylic acid 5-amino-6-oxo-2,3-piperidinedicarboxylic acid 5-amino-6-oxo-2,4-piperidinedicarboxylic acid 5-amino-6-oxo-2,3-piperidinedicarboxylic acid 5-amino-6-oxo[2S-(2a,4β3,5α)]-2,4-piperidinedicarboxylic acid (2S,4R)-2,4-pyrrolidinedicarboxylic acid (2S-cis)-2,4-pyrrolidinedicarboxylic acid 2-amino-1H-imidazole-4,5-dicarboxylic acid 2,5-pyrrolidinedicarboxylic acid 4-amino-3,5-isothiazoledicarboxylic acid 1-methyl-1H-pyrazole-3,5-dicarboxylic acid 7-(diethylamino)-2-oxo-2H-1-benzopyran-3,4-dicarboxylic acid 3,4-diethyl-1H-pyrrole-2,5-dicarboxylic acid 1-phenyl-1H-pyrrole-3,4-dicarboxylic acid cis-2,3-piperazinedicarboxylic acid 2,3-piperazinedicarboxylic acid 2,5-piperazinedicarboxylic acid 2,6-piperazinedicarboxylic acid 2-amino-3,5-pyridinedicarboxylic acid 2-methylpyrrole-3,4-dicarboxylic acid 4-(methylamino)-2,6-pyridinedicarboxylic acid 2-amino-6-methyl-3,4-pyridinedicarboxylic acid 5-amino-2-methyl-3,4-pyridinedicarboxylic acid 2-amino-6-methyl-3,5-pyridinedicarboxylic acid 2,5-dimethylpyrrole-3,4-dicarboxylic acid 2,5-dimethylpyrrole-3,4-dicarboxylic acid 2-amino-6-hydroxy-3,5-pyridinedicarboxylic acid 2,4-pyrrolidinedicarboxylic acid 1H-indole-2,4-dicarboxylic acid 1H-indole-2,6-dicarboxylic acid 1H-indole-2,5-dicarboxylic acid 5-phenyl-2,4-pyrrolidinedicarboxylic acid 5-methyl-2,4-pyrrolidinedicarboxylic acid trans-2,4-azetidinedicarboxylic acid cis-2,4-azetidinedicarboxylic acid 3,5-piperidinedicarboxylic acid 2,3-pyrrolidinedicarboxylic acid 2,3-azetidinedicarboxylic acid 3,4-pyrrolidinedicarboxylic acid 2,3-dihydro-6H-1,4-dioxino[2,3-c]pyrrole-5,7-dicarboxylic acid 1H-imidazole-2,4-dicarboxylic acid 1-butyl-1H-pyrrole-2,3-dicarboxylic acid 3-amino-1-oxide-2,4-pyridinedicarboxylic acid 2,3-dihydro-5-phenyl-1H-pyrrolizine-6,7-dicarboxylic acid 3a,4,5,9b-tetrahydro-3H-cyclopenta[c]quinoline-4,6-dicarboxylic acid 3a,4,5,9b-tetrahydro-3H-cyclopenta[c]quinoline-4,8-dicarboxylic acid 2,3-dihydro-1H-imidazole-4,5-dicarboxylic acid 5-amino-6-methyllutidinic acid 1H-indole-3,7-dicarboxylic acid 3,3-dimethyl-2,6-piperidinedicarboxylic acid 1-butyl-2,5-pyrrolidinedicarboxylic acid 1H-indole-4,6-dicarboxylic acid 1-(phenylmethyl)-3,4-pyrrolidinedicarboxylic acid 3-(carboxymethyl)-1H-indole-2,6-dicarboxylic acid 3,4-bis(2,2,2-trifluoroethyl)-1H-pyrrole-2,5-dicarboxylic acid 9-hexyl-9H-carbazole-3,6-dicarboxylic acid 3-methyl-5-(1-piperazinylsulfonyl)-2,4-thiophenedicarboxylic acid 2,3,4,9-tetrahydro-1H-carbazole-5,7-dicarboxylic acid 2,3-dimethyl-1H-indole-4,6-dicarboxylic acid 7-amino-1,4-dihydro-4-oxo-3,6-quinolinedicarboxylic acid 5-amino-3-methyl-2,4-thiophenedicarboxylic acid (m-tolylim ino)diacetic acid (o-tolylim ino)diacetic acid D-cystathionine phenethyliminodiacetic acid 2-benzyl-2,2′-iminodiacetic acid L-α-glutamyl-L-alanyl-L-alanine N,N′-dibenzylethylenediaminediacetic acid N-L-γ-glutamyl-D-alanine glycyl-L-glutamylglycine N-(carboxymethyl)-N-(tetrahydro-1,1-dioxido-3-thienyl)glycine N-(2-carboxyethyl)-N-phenyl-beta-alanine N-(carboxymethyl)-N-octylglycine N-(tert-butoxycarbonyl)iminodiacetic acid N-(carboxymethyl)-L-alanine N-(6-aminohexyl)-N-(carboxymethyl)glycine N-(carboxymethyl)-N-tetradecylglycine N-(1-carboxyethyl)-D-alanine N-(carboxymethyl)-D-alanine decyliminodiacetic acid 3,3′-(dimethylhydrazono)bis-propanoic acid N-(carboxymethyl)-N-[2-(2,6-dioxo-4-morpholinyl)ethyl]glycine N-alpha-aspartylglycine N-beta-aspartylglycine N-L-alpha-aspartyl-beta-alanine 3,4-xylylamino-N,N-diacetic acid N-(1-carboxyethyl)alanine N-(carboxymethyl)alanine N,N′-methylenebis-glycine N-(aminomethyl)-N-(carboxymethyl)glycine N-(aminomethyl)-N-(carboxymethyl)glycine 2,2′-(methylhydrazono)bis-acetic acid N-(2-carboxyethyl)-N-(4-methylphenyl)-beta-alanine N-(2-carboxyethyl)-N-(3-methylphenyl)-beta-alanine 3-[(carboxymethyl)amino]alanine D-alpha-aspartyl-D-alanine N-(2-carboxyethyl)-N-(1-oxohexadecyl)-beta-alanine N-(2-carboxyethyl)-N-(1-oxodecyl)-beta-alanine N-(2-carboxyethyl)-N-(1-oxotetradecyl)-beta-alanine amino[(carboxymethyl)thio]acetic acid N,N′-1,6-hexanediylbis-beta-alanine N-(carboxymethyl)-N-phenyl-beta-alanine N-(1-carboxyethyl)-L-alanine L-glutamic acid L-aspartic acid.

The total amount of the mono-, di-, and/or tri-carboxylic acids can vary depending on the type of composition. For example a restructuring composition that is added to chemical treatment composition will typically have a higher amount of the one or more mono-, di-, and/or tri-carboxylic acids because the one more mono-, di-, and/or tri-carboxylic acids become more diluted when the restructuring composition is combined with a chemical treatment composition.

The total amount of mono-, di-, and/or tri-carboxylic acids in a restructuring composition is typically about 1 to about 50 wt. %, based on the total weight of the restructuring composition. In some cases, the total amount of the one or more mono-, di-, and/or tri-carboxylic acids in a restructuring composition is about 1 to about 40 wt. %, about 1 to about 30 wt. %, about 1 to about 25 wt. %, about 1 to about 20 wt. %, about 5 to about 50 wt. %, about 5 to about 40 wt. %, about 5 to about 30 wt. %, about 5 to about 25 wt. %, or about 5 to about 20 wt. %.

When mixed with a hair treatment composition or when the hair restructuring components are already formulated as part of a chemical hair treatment formulation, the total amount of mono-, di-, and/or tri-carboxylic acids will typically be less than the total amount in a restructuring composition. For example, the total amount of mono-, di-, and/or tri-carboxylic acids in a chemical hair treatment composition may be about 0.01 to about 10 wt. %, about 0.01 to about 5 wt. %, about 0.01 to about 4 wt. %, about 0.01 to about 3 wt. %, about 0.1 to about 10 wt. %, about 0.1 to about 5 wt. %, about 0.1 to about 4 wt. %, about 0.1 to about 3 wt. %, about 0.5 to about 4 wt. %, or about 0.5 to about 3 wt. %, based on the total weight of the chemical hair treatment composition.

### Amines

Monamines

Many monoamines are suitable for use herein. Non-limiting examples of monoamines methylamine, ethylamine, isopropylamine and n-propylamine, or of a diamine selected from the group consisting of ethylenediamine, 1,2-propanediamine, 1,3-propanediamine, 1,2-butanediamine, 1,3-butanediamine and 1,4-butanediamine or of an alkanolamine selected from the group consisting of monoethanolamine, 2-aminopropan-1-ol and 1-aminopropan-2-ol, or of a specialty amine selected from the group consisting of 1,2,3-triaminopropane, 1,3-diaminopropan-2-ol, 1,2-diamino-propan-3-ol, 1-aminopropanediol, 2-am inopropanediol, glucosamine and isomaltine and/or piperazine, or of a piperazine derivative selected from the group consisting of 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine

The monoamine may be an alkyl monoamine of the present disclosure are amine compounds having one amino group. Non-limiting examples of alkyl monoamines include aliphatic amine compounds corresponding to the following formula and their salts:

RN(R′)₂(IA)

wherein R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and the groups R′, which may be identical or different, represent H or a hydrocarbon radical containing less than 6 carbon atoms. In addition, the groups R′, which may be identical or different, are linear or branched, acyclic or cyclic, saturated or unsaturated, substituted or unsubstituted. In some cases, the groups R′, which may be identical or different, are H or a methyl group.

In some cases, alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

Other non-limiting examples of alkyl monoamines include amidoamine compounds corresponding to the following formula and their salts:

RCONHR′N(R″)₂

wherein: R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and R′ is a divalent hydrocarbon radical containing less than 6 carbon atoms, or 2 or 3 carbon atoms, and R″ is H or a hydrocarbon radical containing less than 6 carbon atoms. In addition, R″ is linear or branched, acyclic or cyclic, saturated or unsaturated, substituted or unsubstituted. Typically, R″ is a linear or branched, acyclic alkyl or alkenyl group. In some cases, R″ is H or a methyl group.

Examples of amidoamines include, but are not limited to the following:

oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palm itam idopropyl dimethylamine, stearamidoethyldiethylamine, and mixtures thereof.

The monoamine may be an alkoxylated monoamine. The alkoxylated monoamines are chosen from amine compounds having an amino group and at least one degree of alkoxylation. The alkoxylation is provided by an alkylene oxide group which is often chosen from ethylene oxide and propylene oxide.

Non-limiting examples of suitable alkoxylated monoamines include compounds corresponding to the following formula:

RN[R′CHCH₂O)ₓH][(R′CHCH₂O)_{y}H]

wherein R is a hydrocarbon radical containing at least 6 carbon atoms. R can be linear or branched, acyclic or cyclic, saturated or unsaturated, aliphatic or aromatic, substituted or unsubstituted; x and y, independently of one another, represent numbers of from 0 to 100 provided that the sum of x+y is >0; the groups R′, which may be identical or different, represent hydrogen, or an alkyl group such as a methyl group. Typically, R is a linear or branched, acyclic alkyl or alkenyl group or an alkyl phenyl group; x and y, independently of one another, are each typically a number from 0 to 30. Typically, one R′ group is hydrogen, and the other one is methyl.

Non-limiting examples of alkoxylated monoamines include PEG-2 Cocamine, PEG-3 Cocamine, PEG-5 Cocamine, PEG-10 Cocamine, PEG-15 Cocamine, PEG-20 Cocamine, PEG-2 Lauramine, PEG-12 Palmitamine, PEG-2 Rapeseedamine, PEG-2 Oleamine, PEG-5 Oleamine, PEG-6 Oleamine, PEG-10 Oleamine, PEG-15 Oleamine, PEG-20 Oleamine, PEG-25 Oleamine, and PEG-30 Oleamine. Other examples are alkoxylated derivatives of soyamine, stearamine and tallow amine.

Other non-limiting examples of suitable alkoxylated monoamines include compounds corresponding the following formula:

RNR″[(R′CHCH₂O)ₓH]

wherein R is a hydrocarbon radical containing at least 6 carbon atoms. R can be linear or branched, acyclic or cyclic, saturated or unsaturated, aliphatic or aromatic, substituted or unsubstituted; x represents a number of from 1 to 100; R′ represents hydrogen, or an alkyl group such as in particular a methyl group; and R″ is a hydrogen or a hydrocarbon radical. Typically, R is a linear or branched, acyclic alkyl or alkenyl group or an alkyl phenyl group; x is typically a number from 1 to 30. When R″ is a hydrocarbon radical group, this group may be linear or branched, saturated or unsaturated, substituted or unsubstituted. The hydrocarbon radical represented by R″ may also contain an alkoxylated moiety (as defined by [(R′CHCH₂O)_{y}H]), and/or heteroatoms such as nitrogen. When R″ contains at least one alkoxylated moiety, the total number of alkoxylation in the formula may range from 1 to 120. Examples of alkoxylated monoamines include PEG-3 Tallow Aminopropylamine, PEG-10 Tallow Aminopropylamine, PEG-15 Tallow Aminopropylamine, and PEG-105 Behenyl Propylenediamine.

Additional non-limiting examples of alkoxylated monoamines include compounds corresponding to the following formula:

R(R′CHCH₂O)ₓ(R′CHCH₂O)_{y}NH_{y}

wherein R is a hydrocarbon radical containing at least 6 carbon atoms. R can be linear or branched, acyclic or cyclic, saturated or unsaturated, aliphatic or aromatic, substituted or unsubstituted; x and y, independently of one another, represent numbers of from 0 to 100 with the proviso that the sum of x+y is >0; the groups R′, which may be identical or different, represent hydrogen, or an alkyl group such as in particular a methyl group. Typically, R is a linear or branched, acyclic alkyl or alkenyl group or an alkyl phenyl group; x and y, independently of one another, are each typically a number from 0 to 30. Examples of alkoxylated monoamines include polyetheramines containing a monoamine group. These polyetheramines are commercially available from Hunstman under the tradename JEFFAMINE (M series such as M-600, M-1000, M-2005 and M-2070) and SURFONAMINE series (B-60, B-100, B-200, L-100, L-200, L-207, L-300).

In some cases, the monamines may be selected from the group consisting of 3-isopropoxypropylamine, 3-methoxypropylamine, tris(hydroxymethyl)aminomethane, 3-ethoxypropylamine, 3-(2-ethylhexyloxy)-propylamine, 2-(2-aminoethoxy)ethanol, 3-butoxypropylamine, and monoethanolamine, and mixtures thereof;

Diamines

Non-limiting examples of diamines that may be useful may be primary amines and secondary amines. The diamine can include both primary and secondary amine groups. Optional diamines may include at least one ethylene oxide group. For example, between 1 and 4 ethylene oxide groups can be present in the diamine. The diamine may optionally include propylene oxide groups. For example, between 1 and 4 propylene oxide groups can be present in the diamine. Non-limiting examples of diamines include 4,9-dioxadodecane-diamine; 4, 7, 10-trioxa-1,13-tridecanediamine; ethylenediamino; polyoxypropylene diamine; polyethylene glycol diamine; triethylene glycol diamine (2OE); n-(2-hydroxyethyl)-ethylenediamine; 1,3-diaminopropane; 1,7-diaminoheptane; 1,4-diaminobutane; 1,2-diaminopropane; 1,6-diaminohexane; 1,11-diamino-3,6,9-trioxaundecane; 1,5-diaminopentane; polyoxyethylene diamine; 2,2-dimethyl-1,3-propanediamine; 2,2-bis(aminoethoxy)propane; 4,7,10-trioxa-1,13-tridecanediamine; 1,3-diaminopentane; 4,7,10-trioxa-1,13; 1,5-diamino-2-methylpentane; (3s,4s)-(-)-3,4-hexanediamine dihydrochloride; 1,9-diaminononane, and mixtures thereof.

In some cases, diamines may be selected from the group consisting of 4,9-dioxadodecane-diamine, 4, 7, 10-trioxa-1,13-tridecanediamine, ethylenediamino, polyoxypropylene diamine, polyethylene glycol diamine, triethylene glycol diamine (2OE); n-(2-hydroxyethyl)-ethylenediamine; 1,3-diaminopropane, 1,7-diaminoheptane, 1,4-diaminobutane, 1,2-diaminopropane, 1,6-diaminohexane, 1,11-diamino-3,6,9-trioxaundecane, 1,5-diaminopentane, polyoxyethylene diamine, 2,2-dimethyl-1,3-propanediamine, 2,2-bis(aminoethoxy)propane, 4,7,10-trioxa-1,13-tridecanediamine, 1,3-diaminopentane, 4,7,10-trioxa-1,13; 1,5-diamino-2-methylpentane, (3s,4s)-(−)-3,4-hexanediamine dihydrochloride, 1,9-diaminononane, and mixtures thereof.

Polyamines

Polyamines have more than two amino groups. In some cases, the composition of the instant disclosure may include one or more polyamines, but in some cases, the compositions are free or essentially free of polyamines. The polyamine may be, for example, aminated polysaccharides comprising multiple amino groups, such as, for example, hydrolysates of aminated polysaccharides.

The polyamine may also be a polymer comprising multiple amino groups including homopolymers, copolymers, and terpolymers.

In some cases, polyamines are chosen from polyethyleneimines. Polyethyleneimines may optionally be substituted. Non-limiting examples of polyethyleneimines which may be used include LUPASOL products commercially available from BASF. Suitable examples of LUPASOL polyethyleneimines include LUPASOL PS, LUPASOL PL, LUPASOL PR8515, LUPASOL G20, LUPASOL G35 as well as LUPASOL SC Polythyleneimine Reaction Products (such as LUPASOL SC-61B, LUPASOL SC-62J, and LUPASOL SC-86X). Other non-limiting examples of polyethyleneimines which may be used in the composition according to the present invention are the EPOMIN products commercially available from Aceto. Suitable examples of EPOMIN polyethyleneimines include EPOMIN SP-006, EPOMIN SP-012, EPOMIN SP-018, and EPOMIN P-1000. Suitable polyamines s also be chosen from polyvinylamines. Examples thereof include LUPAMINES 9095, 9030, 9010, 5095, 1595 from BASF.

The polyamine compounds can also be substituted. An example of such a compound is PEG-15 Cocopolyamine from Cognis.

In some cases, the polyamine is chosen from proteins and protein derivatives. Non-limiting examples of suitable proteins and protein derivatives f include those listed at pages 1701 to 1703 of the C.T.F.A. International Cosmetic Ingredient Dictionary and Handbook, 8^{th} edition, vol. 2, (2000), which is incorporated herein by reference in its entirety. In some cases, the at least one polyamine is chosen from wheat protein, soy protein, oat protein, collagen, and keratin protein.

The polyamine may be an alkoxylated polyamine. The alkoxylated polyamines may be chosen from amine compounds having at least two amino groups and at least one degree of alkoxylation. The alkoxylation is provided by an alkylene oxide group which may be chosen from ethylene oxide and propylene oxide. Non-limiting examples of suitable alkoxylated polyamines include compounds corresponding to the following formula:

NH₂R(R′CHCH₂O)ₓ(R′CHCH₂O)_{y}(R′CHCH₂O)_{z-}—RNH₂

wherein R represents a —CH2—, —CH₂CH₂—, —CHCH₃— or —C(CH₃)₂ group, or a hydrocarbon radical containing at least 3 carbon atoms that is linear or branched, acyclic or cyclic, saturated or unsaturated, aliphatic or aromatic, substituted or unsubstituted; x, y, and z independently of one another, represent numbers of from 0 to about 100; R′ represents hydrogen, or an alkyl group, preferably a methyl group; and The sum of x+y+z is at least 1. In some cases, R is a linear or branched, acyclic alkyl or alkenyl group or an alkyl phenyl group; x, y, and z independently of one another, preferably represent numbers ranging from 2 to 100.

Non-limiting examples of the alkoxylated polyamines include, for example, tetradecyloxypropyl-1,3-diaminopropane; a C₁₂-₁₄ alkyl oxypropyl-1,3-diaminopropane; a C_{12 -15} alkyloxypropyl amine and other similar materials that are commercially available from Tomah under the tradename of TOMAH DA-17. Other examples of alkoxylated polyamines are diamine compounds belonging to the Jeffamine series such as the JEFFAMINE D and JEFFAMINE ED series available from Huntsman Corporation, Salt Lake City, Utah. Examples of these Jeffamine compounds are JEFFAMINE D230, JEFFAMINE D400, JEFFAMINE D2000, JEFFAMINE D4000, JEFFAMINE HK-511, JEFFAMINE ED600, JEFFAMINE ED900, and JEFFAMINE ED2003. JEFFAMINE D series compounds are amine terminated PPGs (polypropylene glycols) and JEFFAMINE ED series compounds are polyether diamine based with a predominantly PEG (polyethylene glycol) backbone.

Other non-limiting examples of suitable alkoxylated polyamines in the diamine form include compounds corresponding to the following formula:

NH₂(CH₂)ₓOCH₂CH₂O(CH₂)ₓNH₂

wherein x is 2 or 3.

Examples of alkoxylated polyamines are diamine compounds belonging to the JEFFAMINE series available from Huntsman Corporation, Salt Lake City, Utah, such as JEFFAMINE EDR148, and JEFFAMINE EDR176.

Additional non-limiting examples of alkoxylated polyamines in the triamine form include compounds corresponding to the following formula:

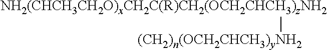

wherein R is hydrogen, —CH₂ or —C₂H₅, n=0 or 1, and x, y, and z independently of one another, represent numbers of from 0 to 100 and the sum of x+y+z is at least 1.

Examples of alkoxylated polyamines are triamine compounds belonging to the JEFFAMINE series such as the JEFFAMINE T series available from Huntsman Corporation, Salt Lake City, Utah. Examples of the JEFFAMINE T series compounds are JEFFAMINE T403, JEFFAMINE T3000, and JEFFAMINE T5000. JEFFAMINE T series compounds are triamines made by reacting PO with a triol initiator followed by aminating the terminal hydroxyl groups.

The total amount of the one or more amines may vary depending on the type of composition. In some cases, the total amount of the one or more amines is about 0.1 to about 35 wt. %, based on the total weight of the composition. In some cases, the total amount of the one or more amines is about 0.1 to about 30 wt. %, about 0.1 to about 25 wt. %, about 0.1 to about 20 wt. %, about 0.1 to about 15 wt. %, about 0.1 to about 10 wt. %, about 0.5 to about 35 wt. %, about 0.5 to about 30 wt. %, about 0.5 to about 25 wt. %, about 0.5 to about 20 wt. %, about 0.5 to about 15 wt. %, about 0.5 to about 10 wt. %, about 1 to about 35 wt. %, about 1 to about 30 wt. %, about 1 to about 25 wt. %, about 1 to about 20 wt. %, about 1 to about 15 wt. %, about 1 to about 10 wt. %, or about 3 to about 8 wt. %.

The total amount of the one or more amines may vary depending on the type of composition. For example a restructuring composition that is added to chemical treatment composition will typically have a higher amount of the one or more amines because the one more amines become more diluted when the restructuring composition is combined with a chemical treatment composition.

In some cases, the total amount of the one or more amines in a restructuring composition is about 0.1 to about 35 wt. %, based on the total weight of the restructuring composition. In some cases, the total amount of the one or more amines is about 0.1 to about 30 wt. %, about 0.1 to about 25 wt. %, about 0.1 to about 20 wt. %, about 0.1 to about 15 wt. %, about 0.1 to about 10 wt. %, about 0.5 to about 35 wt. %, about 0.5 to about 30 wt. %, about 0.5 to about 25 wt. %, about 0.5 to about 20 wt. %, about 0.5 to about 15 wt. %, about 0.5 to about 10 wt. %, about 1 to about 35 wt. %, about 1 to about 30 wt. %, about 1 to about 25 wt. %, about 1 to about 20 wt. %, about 1 to about 15 wt. %, about 1 to about 10 wt. %, or about 3 to about 8 wt. %.

In situation where the components of the restructuring composition are already formulated as part of a chemical hair treatment composition, the total amount of the one or more amines may be about 0.01 to about 8 wt. %, about 0.01 to about 4 wt. %, 0.01 to about 3 wt. %, about 0.01 to about 2 wt. %, about 0.05 to about 8 wt. %, about 0.05 to about 4 wt. %, 0.05 to about 3 wt. %, or about 0.05 to about 2 wt. %, based on the total weight of the chemical treatment composition.

### Polymeric Acid and Polymeric Acid Anhydride Compounds

The polymeric acid and polymeric acid anhydride compounds of the present disclosure are polymers derived from at least one of carboxylics acid, sulfonic acids, and phosphoric acids, and generally have a number-average molecular mass ranging from 500 to 5,000,000.

The polymeric acid and polymeric acid anhydride compounds may be chosen from aliphatic, cycloaliphatic and aromatic polyacids, unsaturated polyacids, and associative polyacids. The carboxylic groups may be provided, for example, by unsaturated monocarboxylic or dicarboxylic acid monomers such as those corresponding to the following formula:

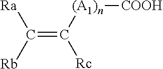

in which: n is an integer ranging from 0 to 10, A₁ is chosen from methylene groups, optionally connected to the carbon atom of the unsaturated group, or to the neighboring methylene group when n is greater than 1, via a hetero atom such as oxygen and sulfur, Rₐ is chosen from hydrogen phenyl groups, and benzyl groups, R_{b} is chosen from hydrogen, (C₁₋₄)alkyl groups, for example, methyl and ethyl, and carboxyl groups, and R_{c} is chosen from hydrogen, lower alkyl groups, —CH2—COOH groups, phenyl groups, and benzyl groups.

Suitable polymers comprising carboxylic groups include, for example:

A) acrylic or methacrylic acid homo- and copolymers, and salts thereof, for example, the products sold under the names VERSICOL E and K by Allied Colloid and Ultrahold by BASF, copolymers of acrylic acid and of acrylamide, and sodium salts of polyhydroxycarboxylic acid;

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No. 1 222 944 and German Patent Application No. 2 330 956, the copolymers of this type comprising an optionally N-alkylated and/or hydroxyalkylated acrylamide unit in their chain as described, for example, in Luxembourg Patent Application Nos. 75370 and 75371. Other examples include copolymers of acrylic acid and of C1-C4 alkyl methacrylate and terpolymers of vinylpyrrolidone, of acrylic acid and of methacrylate of C1-C20 alkyl, for example of lauryl, such as the product sold by ISP under the name ACRYLIDONE LM and methacrylic acid/ethyl acrylate/tert-butyl acrylate terpolymers such as the product sold under the name LUVIMER 100 P by BASF;

C) copolymers derived from crotonic acid such as those comprising vinyl acetate or propionate units in their chain and optionally other monomers such as allylic esters or methallylic esters, vinyl ether or vinyl ester of a linear or branched saturated carboxylic acid with a long hydrocarbon-based chain such as those comprising at least 5 carbon atoms, it being possible for these polymers optionally to be grafted and crosslinked, or alternatively another vinyl, allylic or methallylic ester monomer of an alpha - or beta-cyclic carboxylic acid. Such polymers are described, for example, in French Patent Nos. 1 222 944, 1 580 545, 2 265 782, 2 265 781, 1 564 110, and 2 439 798. A non-limiting example of a suitable commercial product is the resin 28-29-30 sold by National Starch;

D) copolymers derived from C4-C8 monounsaturated carboxylic acids chosen from: copolymers comprising (i) at least one entity chosen from maleic, fumaric, and itaconic acids and anhydrides and (ii) at least one monomer chosen from vinyl esters, vinyl ethers, vinyl halides, phenylvinyl derivatives, and acrylic acid and its esters, the acid functions of these copolymers optionally being monoesterified or monoamidated. Such polymers are described, for example, in U.S. Pat. Nos. 2,047,398, 2,723,248, and 2,102,113 and British Patent No. 839 805. Suitable commercial products include, for example, those sold under the names GANTREZ S and ES by ISP, copolymers comprising (i) at least one unit chosen from maleic, citraconic, and itaconic acid units and (ii) at least one monomer chosen from allylic or methallylic esters optionally comprising at least one group chosen from acrylamide, methacrylamide, and alpha-olefin groups, acrylic esters, methacrylic esters, acrylic acids, methacrylic acids, and vinylpyrrolidone in their chain, the acid functions of these copolymers optionally being monoesterified or monoamidated. These polymers are described, for example, in French Patent Nos. 2 350 384 and 2 357 241;

E) polyacrylamides comprising carboxylate groups; and

F) anionic polyurethanes, such as the product sold by BASF under the name LUVISET PUR.

The polymeric acid and polymeric acid anhydride compounds comprising sulfonic groups may be polymers comprising vinylsulfonic, styrenesulfonic, naphthalenesulfonic, and/or acrylamidoalkylsulfonic units. These polymers may be chosen, for example, from: polyvinylsulfonic acid salts having a molecular weight ranging from 1000 to 100,000, as well as copolymers with an unsaturated comonomer such as acrylic or methacrylic acids and their esters, as well as acrylamide and its derivatives, vinyl ethers, and vinylpyrrolidone;

polystyrenesulfonic acid salts, such as the sodium salts sold, for example, under the name FLEXAN 130 by National Starch. These compounds are described, for example, in French Patent No. 2 198 719; polyacrylamidesulfonic acid salts, such as those mentioned in U.S. Pat. No. 4,128,631, for example, polyacrylamidoethylpropanesulfonic acid.

The polymeric acid and polymeric acid anhydride compounds may be chosen from those of grafted silicone type comprising a polysiloxane portion and a portion consisting of a non-silicone organic chain, one of the two portions constituting the main chain of the polymer, the other being grafted onto the said main chain. These polymers are described, for example, in European Patent Application Nos. 0 412 704, 0 412 707, 0 640 105, and 0 582 152, International Patent Application Publication Nos. WO 95/00578 and WO 93/23009, and U.S. Pat. Nos. 4,693,935, 4,728,571, and 4,972,037. Such polymers may include, for example, copolymers that can be obtained by radical polymerization from a monomer mixture comprising:

a) 50 to 90 percent by weight of tert-butyl acrylate;

b) 0 to 40 percent by weight of acrylic acid; c) 5 to 40 percent by weight of silicone macromer of formula (II):

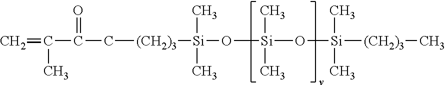

with v being a number from 5 to 700; the weight percentages being calculated relative to the total weight of the monomers.

Other examples of grafted silicone polymers include, but are not limited to, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

In some cases, functionalized silicone or non-silicone polyurethanes may also be used as film-forming polymers. Examples of suitable polyurethanes include those disclosed in European Patent Nos. 0 751 162, 0 637 600, 0 648 485, 0 619 111, and 0 656 021, French Patent No. 2 743 297, and International Patent Application Publication No. WO 94/03510.

Also, the polymers may be chosen from acrylic acid copolymers, such as the acrylic acid/ethyl acrylate/N-tert-butylacrylamide terpolymers sold under the name ULTRAHOLD Strong by BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by National Starch, polymers derived from maleic, fumaric, or itaconic acids with isobutylene, vinyl esters, vinyl ethers, vinyl halides, phenylvinyl derivatives and acrylic acid and esters thereof, such as the methyl vinyl ether/monoesterified maleic acid copolymers sold, for example, under the name GANTREZ by ISP, the copolymers of methacrylic acid and of methyl methacrylate sold under the name EUDRAGIT L by Rohm Pharma, the copolymers of methacrylic acid and of ethyl acrylate sold under the name LUVIMER MAEX and MAE by BASF, the vinyl acetate/crotonic acid copolymers and the vinyl acetate/crotonic acid copolymers grafted with polyethylene glycol sold under the name ARISTOFLEX A by BASF, and the polyurethane LUVISET PUR sold by BASF.

The polymers may, in at least some cases, be chosen from the methyl vinyl ether/monoesterified maleic acid copolymers sold under the name GANTREZ ES 425 by ISP, the acrylic acid/ethyl acrylate/N-tert-butylacrylamide terpolymers sold under the name ULTRAHOLD Strong by BASF, the copolymers of methacrylic acid and of methyl methacrylate sold under the name EUDRAGIT L by Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name LUVIMER MAEX and MAE by BASF, the vinylpyrrolidone/acrylic acid/lauryl methacrylate terpolymers sold under the name ACRYLIDONE LM by ISP, and the polyurethane LUVISET PUR sold by BASF.

The polymeric acid and polymeric acid anhydride compounds of the present disclosure may also include, for example, those polymers comprising units B and C distributed randomly in the polymer chain, in which B denotes a unit derived from a monomer comprising at least one basic nitrogen atom and C denotes a unit derived from an acid monomer comprising at least one group chosen from carboxylic and sulfonic groups, or alternatively B and C may denote groups derived from monomers chosen from carboxybetaine and sulfobetaine zwitterionic monomers.

B and C may also denote a cationic polymer chain comprising primary, secondary, tertiary, and/or quaternary amine groups, in which at least one of the amine groups bears a carboxylic or sulfonic group connected via a hydrocarbon-based group or alternatively B and C form part of a chain of a polymer containing an alpha-dicarboxylic ethylene unit in which one of the carboxylic groups has been made to react with a polyamine containing one or more primary or secondary amine groups.

Non-limiting examples of polymers corresponding to the definition given above include:

(1) polymers resulting from the copolymerization of a monomer derived from a vinyl compound comprising a carboxylic group such as acrylic acid, methacrylic acid, maleic acid, and alpha-chloroacrylic acid, and a basic monomer derived from a substituted vinyl compound comprising at least one basic atom (for example, an amino function), such as dialkylaminoalkyl methacrylate and acrylate, and dialkylam inoalkylmethacrylam ides and dialkylam inoalkylacrylam ides. Such compounds are described, for example, in U.S. Pat. No. 3,836,537. A non-limiting example of a commercially available product is the sodium acrylate/acrylamidopropyl trimethylammonium chloride copolymer sold under the name POLYQUART KE 3033 by Henkel.

The vinyl compound may also be a dialkyldiallylammonium salt such as diethyldiallylammonium chloride. The copolymers of acrylic acid and of the latter monomer are sold, for example, under the names MERQUAT 280, MERQUAT 295, and MERQUAT Plus 3330 by Calgon.

(2) polymers comprising units derived from: a) at least one monomer chosen from acrylam ides and methacrylam ides substituted on the nitrogen atom with an alkyl group, b) at least one acidic comonomer comprising at least one reactive carboxylic group, and c) at least one basic comonomer such as esters containing primary, secondary, tertiary, and quaternary amine substituents of acrylic and methacrylic acids and the product of quaternization of dimethylaminoethyl methacrylate with dimethyl or diethyl sulfate.

The N-substituted acrylam ides or methacrylam ides may include compounds in which the alkyl groups comprise from 2 to 12 carbon atoms, for example, N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers may be chosen, for example, from acrylic acid, methacrylic acid, crotonic acid, itaconic acid, maleic acid, fumaric acid, and alkyl monoesters, comprising from 1 to 4 carbon atoms, of maleic or fumaric acids and anhydrides. Suitable basic comonomers include, for example, aminoethyl, butylaminoethyl, N,N′-dimethylaminoethyl, and N-tert-butylaminoethyl methacrylates. A further non-limiting example is the copolymer whose CTFA (4th edition, 1991) name is octylacrylamide/acrylates/butylaminoethyl methacrylate copolymer, such as the products sold under the names AMPHOMER and LOVOCRYL 47 by National Starch.

(3) crosslinked and acylated polyamino amides partially or totally derived from polyamino amides of the following formula:

CO—R₁₀—CO—Z (III)

in which: R₁₀ is chosen from divalent groups derived from a saturated dicarboxylic acid, mono- or dicarboxylic aliphatic acids comprising an ethylenic double bond, esters of lower alkanols, comprising from 1 to 6 carbon atoms, of these acids, and groups derived from the addition of any one of said acids to a bis(primary) or bis(secondary) amine, and Z is a group derived from a bis(primary), mono- or bis(secondary) polyalkylene-polyamine and, in at least one embodiment, may represent:
a) in an amount ranging from 60 to 100 mol %, the group

—NH(CH₂)—NHₚ

where x=2 and p=2 or 3, or alternatively, x=3 and p=2 this group being derived from diethylenetriamine, from triethylenetetraamine, or from dipropylenetriamine;
b) in an amount ranging from 0 to 40 mol %, the group (IV) above in which x=2 and p=1 and which is derived from ethylenediamine, or the group derived from piperazine:

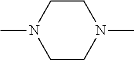

c) in an amount ranging from 0 to 20 mol %, the —NH—(CH₂)₆)—NH— group derived from hexamethylenediamine, these polyamino amides being crosslinked by addition reaction of a difunctional crosslinking agent chosen from epihalohydrins, diepoxides, dianhydrides, and bis-unsaturated derivatives, using from 0.025 to 0.35 mol of crosslinking agent per amine group of the polyamino amide and acylated by the action of an entity chosen from acrylic acid, chloroacetic acid, an alkane sultone, and salts thereof.

The saturated carboxylic acids may be chosen from acids comprising from 6 to 10 carbon atoms, such as adipic acid, 2,2,4-trimethyladipic acid, 2,4,4-trimethyladipic acid, terephthalic acid, and acids comprising an ethylenic double bond, for instance acrylic acid, methacrylic acid, and itaconic acid.

The alkane sultones used in the acylation may be chosen, for example, from propane sultone and butane sultone, and the salts of the acylating agents may be chosen from sodium and potassium salts.

(4) polymers comprising zwitterionic units of the following formula:

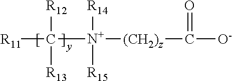

in which: R₁ is a polymerizable unsaturated group, for example, an acrylate, methacrylate, acrylamide, and methacrylamide group, y and z, which may be identical or different, are integers ranging from 1 to 3, R₁₂ and R₁₃, which may be identical or different, are chosen from hydrogen and methyl, ethyl, and propyl groups, and R₁₄ and R₁₅, which may be identical or different, are chosen from hydrogen and alkyl groups such that the sum of the carbon atoms in Ry and Ry does not exceed 10.

The polymers comprising such units may also contain units derived from nonzwitterionic monomers such as dimethylaminoethyl acrylate, dimethylaminoethyl methacrylate, diethylaminoethyl acrylate, diethylaminoethyl methacrylate, alkyl acrylates, alkyl methacrylates, acrylamides, methacrylamides, and vinyl acetate. Non-limiting example of such copolymers include butyl methacrylate/N,N-dimethylcarboxyaminoethyl methacrylate copolymers.

(5) polymers derived from chitosan comprising monomer units chosen from units of formula (D)-(F):

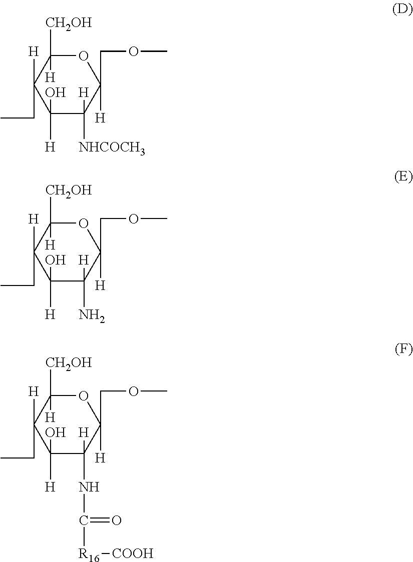

the unit (D) being present in an amount ranging from 0% to 30%, the unit (E) in an amount ranging from 5% to 50%, and the unit (F) in an amount ranging from 30% to 90%, it being understood that, in this unit (F), R₁₆ is a group of formula:

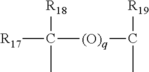

in which, if q=0, R₁₇, R₁₈, and R₁₉, which may be identical or different, are chosen from hydrogen, methyl, hydroxyl, acetoxy, and amino residues, monoalkylamine residues and dialkylamine residues that are optionally interrupted by at least one nitrogen atom and/or optionally substituted with at least one group chosen from amine, hydroxyl, carboxyl, alkylthio, and sulfonic groups, alkylthio residues in which the alkyl group comprises an amino residue, at least one of the groups R₁₇, R₁₈, and R₁₉ being, in this case, a hydrogen atom; or, if q=1, R₁₇, R₁₈, and R₁₉ are each hydrogen atoms, as well as the acid and base addition salts of these compounds.

(6) polymers described, for example, in French Patent No. 1 400 366 and comprising the repeating unit of the following formula:

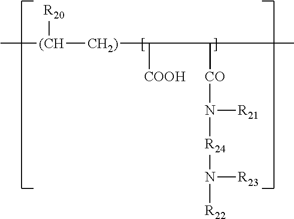

in which: R₂₀ is chosen from hydrogen, CH₃O, CH₃CH₂O, and phenyl groups, R₂₁ is chosen from hydrogen and lower alkyl groups such as methyl and ethyl, R₂₂ is chosen from hydrogen and C₁-C₆ lower alkyl groups such as methyl and ethyl, R₂₄ is chosen from —CH₂—CH₂—, —CH₂—CH₂—CH₂—, and —CH₂—CH(CH₃)— groups, and R₂₃ is chosen from C1-C6 lower alkyl groups such as methyl and ethyl and groups corresponding to the formula: —R₂₄—N(R₂₂)₂—, wherein R₂₄ is chosen from —CH₂—CH₂—, —CH₂—CH₂—CH₂—, and —CH₂—CH(CH₃)— groups, and R₂₂ has the definition given above.

(7) polymers derived from the N-carboxyalkylation of chitosan, such as N-carboxymethylchitosan and N-carboxybutylchitosan.

(8) amphoteric polymers of the type -D-X-D-X chosen from:
a) polymers obtained by the action of chloroacetic acid or sodium chloroacetate on compounds containing at least one unit of formula:

-D-X-D-X-D-

wherein D denotes a group

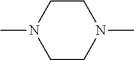

and X is chosen from the symbols E or E′, wherein E or E′, which may be identical or different, are chosen from divalent groups that are alkylene groups comprising a straight or branched chain comprising up to 7 carbon atoms in the main chain, which is unsubstituted or substituted with hydroxyl groups and which may contain, in addition to oxygen, nitrogen, and sulfur atoms, 1 to 3 aromatic and/or heterocyclic rings; the oxygen, nitrogen, and sulfur atoms being present in the form of an entity chosen from ether, thioether, sulfoxide, sulfone, sulfonium, alkylamine, and alkenylamine groups, hydroxyl, benzylamine, amine oxide, quaternary ammonium, amide, imide, alcohol, ester, and/or urethane groups.
b) polymers of formula:

-D-X-D-X—

wherein D denotes a group

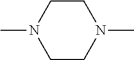

and X is chosen from the symbols E and E′, and at least once E′; E having the meaning given above and E′ being a divalent group that is an alkylene group with a straight or branched chain having up to 7 carbon atoms in the main chain, which is unsubstituted or substituted with at least one hydroxyl group and containing at least one nitrogen atom, the nitrogen atom being substituted with an alkyl chain that is optionally interrupted by an oxygen atom and comprising at least one carboxyl function or at least one hydroxyl function and betainized by reaction with an entity chosen from chloroacetic acid and sodium chloroacetate.

(9) (C1-C5) alkyl vinyl ether/maleic acid copolymers partially modified by semiamidation with an N,N-dialkylaminoalkylamine such as N,N-dimethylam inopropylamine or by semiesterification with an N,N-dialkylaminoalkynol. These copolymers may also contain other vinyl comonomers such as vinylcaprolactam.

The polyacid of the present disclosure may also be chosen from silicone acids such as polysilicone-8 (3M brand silicones “Plus” polymer VS 80, commercially available from 3M company) which has the structure:

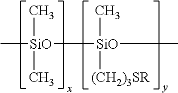

where R represents the acrylates copolymer radical.

In some instances, the polymeric acid and polymeric acid anhydride compound is chosen from VA/crotonates copolymer (and) isopropyl alcohol, Butyl ester of PVM/MA copolymer, isopropyl ester of PVM/MA copolymer, Ethyl ester of PVM/MA copolymer, VA/crotonates/vinyl neodecanoate copolymer, VA/crotonates copolymer, Ethylene/acrylic acid copolymer, VA/vinyl butyl benzoate/crotonates copolymer, Acrylates/octylacrylamide copolymer, Acrylates/t-Butylacrylamide copolymer, VP/acrylates/lauryl methacrylate copolymer, Styrene/acrylates copolymer, Acrylates copolymer, Polyacrylate-3, Carbomer, Acrylates/C10-30 alkyl acrylate crosspolymer and mixtures thereof.

In some cases, the polymeric acid and polymeric acid anhydride compound is one more maleic acid and/or maleic anhydride polymers. The one or more maleic acid and/or maleic anhydride polymers may be homopolymers and/or copolymers. Non-limiting examples of maleic anhydride copolymers include poly(styrene-alt-maleic anhydride), poly(methyl vinyl ether-alt-maleic anhydride), poly(ethylene-g-maleic anhydride), poly(isobutylene-alt-maleic anhydride), polyisoprene-g-maleic anhydride, poly(maleic anhydride-alt-1-octadecene), poly(ethylene-co-ethyl acrylate-co-maleic anhydride), polyethylene-graft-maleic anhydride, and mixtures thereof.

Non-limiting examples maleic acid copolymers selected from the group consisting of butyl ester of ethylene/MA copolymer, butyl ester of PVM/MA copolymer, calcium/sodium salt of PVM/MA copolymer, ethyl ester of PVM/MA copolymer, ethylene/MA copolymer, isobutylene/MA copolymer, isopropyl ester of PVM/MA copolymer, octadecene/MA copolymer, polyethylene/isopropyl maleate/MA copolymer, polyvinyl methyl ether (PVM)/MA copolymer, PVM/MA decadiene crosspolymer, sodium C4-C12 olein/maleic acid copolymer, sodium isooctylene/MA copolymer, sodium MA/diisobutylene copolymer, sodium PVM/MA/decadiene crosspolymer, stearylvinyl ether/MA copolymer, styrene/MA copolymer, and mixtures thereof.

The maleic acid polymer may include homopolymers and copolymers of maleic acid. In one embodiment, the maleic acid polymer has a molecular weight of between about 500 and about 5,000 g/mol. Examples of suitable maleic acid polymers include AQUATREAT AR-801 available from Alco Chemical, Chattanooga, Tenn., OPTIDOSE 4210 available from Dow and BELCLENE 200 available from Houghton Chemical Corporation.

In some cases, the maleic acid polymer is a maleic/methylvinyl ether copolymer (PVM/MA copolymer), which may have a molecular weight of between about 125,000 g/mol and about 800,000 g/mol, and/or a polydispersity index of between about 2 and 6. The maleic and methylvinyl ether monomer segments may be randomly arranged and/or have alternating segments such that the resulting maleic/methylvinyl ether copolymer has the following general structure:

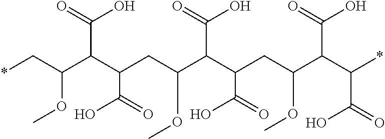 [SMILES: *CC(OC)C(C(=O)O)C(CC(OC)C(C(=O)O)C(CC(OC)C(C(=O)O)C(*)C(=O)O)C(=O)O)C(=O)O]

Examples of suitable maleic/methylvinyl ether copolymers are commercially available from ISP under the tradename “GANTREZ,” as well as other suppliers such as SigmaAldrich. Mixtures of maleic acid polymers and maleic/methylvinyl ether copolymers may also be employed.

The polymeric acid and polymeric acid anhydride compound, in some cases, can be a poly(maleic anhydride/alkene-1) compound. The “maleic anhydride” component in this case includes α,β,-olefinically unsaturated dicarboxylic acid anhydride comonomers represented by the structural formula:

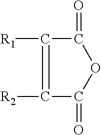

wherein R₁ and R₂ are independently selected from the group consisting of hydrogen, halogen, cyano, and aliphatic and aromatic substituents such as alkyl, aryl, alkaryl, aralkyl, cycloaliphatic, and the like, containing between one and about ten carbon atoms.

Compounds corresponding to the above formula include maleic anhydride, chloromaleic anhydride, 2,3-dichloromaleic anhydride, cyanomaleic anhydride, 2,3-dicyanomaleic anhydride, methylmaleic anhydride, 2,3-dimethylmaleic anhydride, ethylmaleic anhydride, propylmaleic anhydride, butylmaleic anhydride, 2,3-di-n-butylmaleic anhydride, phenylmaleic anhydride, benzylmaleic anhydride, cyclohexylmaleic anhydride, and the like.

The term “alkene-1” is meant to include alphaolefinically unsaturated hydrocarbons containing between 2 and about 6 carbon atoms. Illustrative of preferred alkene-1 comonomers are ethylene, Propylene, isobutylene, butene-1, pentene-1 and hexene-1.

Other maleic anhydride polymers include, but are not limited to, A-C 573 A (ETHYLENE-MALEIC ANHYDRIDE COPOLYMER) from Honeywell, A-C 596 A (PROPYLENE-MALEIC ANHYDRIDE COPOLYMER) from Honeywell, A-C 597 (PROPYLENE-MALEIC ANHYDRIDE COPOLYMER) from Honeywell, ZEMAC copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemical Co., poly (ethylene-co-butyl acrylate-co-maleic anhydride) sold under the trade name of LOTADER (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the LOTADER name, and isobutylene maleic anhydride copolymer sold under the name ACO-5013 by ISP.

The total amount of the one or more The polymeric acid and polymeric acid anhydride compounds will vary depending on the composition. For instance, a restructuring composition that is to be added to another hair treatment composition will have a higher amount of the maleic acid and/or maleic anhydride polymers because it will become more diluted upon mixing with the hair treatment composition. The total amount of the one or more The polymeric acid and polymeric acid anhydride compounds in a restructuring composition may be about 0.01 to about 20 wt. %, about 0.01 to about 10 wt. %, about 0.01 to about 5 wt. %, about 0.01 to about 4 wt. %, about 0.01 to about 3 wt. %, about 0.01 to about 2 wt. %, about 0.1 to about 10 wt. %, about 0.1 to about 5 wt. %, about 0.1 to about 4 wt. %, about 0.1 to about 3 wt. %, or about 0.1 to about 2 wt. %, based on the total weight of the composition.

When mixed with a hair treatment composition or when the hair restructuring components are already formulated as part of a chemical hair treatment formulation, the total amount of the one or more The polymeric acid and polymeric acid anhydride compounds will typically be less than the total amount in a restructuring composition. For example, the total amount of the maleic acid and/or maleic anhydride polymers in a chemical hair treatment composition may be about 0.01 to about 10 wt. %, about 0.01 to about 5 wt. %, about 0.01 to about 3 wt. %, about 0.01 to about 2 wt. %, about 0.01 to about 1 wt. %, about 0.1 to about 15 wt. %, about 0.1 to about 10 wt. %, about 0.1 to about 5 wt. %, about 0.1 to about 3 wt. %, about 0.1 to about 2 wt. %, or about 0.1 to about 1 wt. % based on the total weight of the chemical hair treatment composition.

### Active Agents

Oxidizing Agents

Oxidizing agents may be selected from, for example, peroxides, persulfates, perborates percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. Oxygen in the air may also be employed as an oxidizing agent.

In some cases, the oxidizing agent is a persulfate and/or a monopersulfate such as, for example, potassium persulfate, sodium persulfate, ammonium persulfate, as well as mixtures thereof. In one embodiment, the oxidizing agents in the present disclosure are selected from hydrogen peroxide, potassium persulfate, sodium persulfate, and mixtures thereof.

One or more oxidizing agents are typically included in an oxidizing composition. An oxidizing composition may be a hair lightening or bleaching composition or it may be a neutralizing composition or a developer composition. In some cases, the total amount of the one or more oxidizing agents in an oxidizing composition is essentially 100% (as is the case for some powdered oxidation compositions). In some cases, the total amount of the one or more oxidizing agents is about 1 to about 80 wt. %, about 1 to about 70 wt. %, about 1 to about 60 wt. %, about 1 to about 50 wt. %, about 1 to about 40 wt. %, about 5 to about 80 wt. %, about 5 to about 70 wt. %, about 5 to about 60 wt. %, about 5 to about 50 wt. %, about 5 to about 40 wt. %, about 10 to about 80 wt. %, about 10 to about 70 wt. %, about 10 to about 60 wt. %, about 10 to about 50 wt. %, or about 10 to about 40 wt. %, based on the total weight of the composition.

Reducing Agents

Reducing agents are well known for use in hair care compositions. Typical reducing agents are capable of reducing the disulfide bonds in the hair to produce free thiol groups. Non-limiting examples of suitable reducing agents includethioglycolic acid and thioglycolic acid salts and esters, thiolactic acid and thiolactic acid salts and esters, cysteine thioglycerol, thioglycolic hydrazide, thioglycolamide, glycerol monothioglycolate, sodium metabisulfite, beta-mercaptopropionic acid, N-hydroxyethyl mercapto-acetamide, N-methyl mercapto-acetamide, beta-mercapto-ethylamine, beta-mercaptopropionamide, 2-mercapto-ethanesulfonic acid, dimercaptoadipic acid, dithiothreitol, homocysteinethiolactone, cysteine derivatives, polythiol derivatives formed by the addition of cysteamine onto a maleic anhydride-alkylvinylether copolymer, inorganic sulfites, inorganic bisulfites, cysteamine and its derivatives, dithioerythritol, organic phosphines, and mixtures thereof.

One or more reducing agents may be included in reducing compositions. The total amount of the one or more reducing agents can vary, but in some cases, the total amount of the one or more reducing agents is about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the composition.

Neutralizing Agents

Neutralizing agents are well known for use in hair care compositions. In some cases, after treating hair with compositions of the present disclosure comprising active agents chosen from reducing agents for curling or shaping the hair (as in perming and hair straightening systems), the hair is treated with a neutralizing agent or composition containing a neutralizing agent. For instance, the neutralizing agent may be an oxidizing agent chosen from hydrogen peroxide, urea hydrogen peroxide, alkali metal bromates, or persalts, such as perborates and persulphates. One or more neutralizing agents may be included in neutralizing compositions. The total amount of the one or more neutralizing agents can vary, but in some cases, the total amount of the one or more neutralizing agents is about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the composition.

Non-Reducing Agents for Shaping Hair

Non-reducing agents for shaping hair may be one or more hydroxide compounds, non-hydroxide compounds, or mixtures thereof. For instance, the hydroxide compounds may be alkali metal hydroxides, alkaline-earth metal hydroxides, transition metal hydroxides, and mixtures thereof. Non-limiting examples include of hydroxide compounds include sodium hydroxide, potassium hydroxide, lithium hydroxide, calcium hydroxide, magnesium hydroxide, barium hydroxide, strontium hydroxide, manganese hydroxide, zinc hydroxide, guanidine hydroxide, and mixtures thereof.

Colorants

Before, after, or simultaneously with the hair lightening composition, a color-altering composition may be used. For example, the color-altering composition may be formed by combining a hair lightening composition according to the instant disclosure, a developer composition (typically comprising hydrogen peroxide) and a colorant. Typically, the coloring compositions of the present disclosure include at least one colorant compound chosen from oxidative dye precursors, direct dyes, pigments, and mixtures thereof. The oxidation dyes are generally chosen from one or more oxidation bases optionally combined with one or more couplers. By way of example, the oxidation bases are chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediam ine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(8-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropano-I, N,N′-bis(β-hydroxyethyl)-N,N′-bis(4′-aminophenypethyl)enediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-ethylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetam ido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds such as 2,5-diaminopyridine, 2-(4-methoxyphenyl)amino-3-aminopyridine and 3,4-diaminopyridine, and the addition salts thereof. Other pyridine oxidation bases that are useful in the present disclosure are the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-.quadrature.-hydroxyethoxy-3-amino-pyrazolo[1,5-a]pyridine; 2-(4-dimethylpyperazinium-1-yl)-3-amino-pyrazolo[1,5-a]pyridine; and the addition salts thereof.

More particularly oxidation bases that are useful in the present disclosure are selected from 3-aminopyrazolo-[1,5-a]-pyridines and preferably substituted on carbon atom 2 by:

(a) one (di)(C₁-C₆)(alkyl)amino group wherein said alkyl group can be substituted by at least one hydroxy, amino, imidazolium group;

(b) one heterocycloalkyl group containing from 5 to 7 members chain, and from 1 to 3 heteroatoms, potentially cationic, potentially substituted by one or more (C₁-C₆-alkyl, such as di(C1-C4)alkylpiperazinium; or

(c) one (C₁-C₆)alkoxy potentially substituted by one or more hydroxy groups such as .quadrature.-hydroxyalkoxy, and the addition salts thereof.

Among the pyrimidine derivatives that may be mentioned are compounds such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are compounds such as 4,5-diamino-1-methyl-pyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4, 5-diamino-1-ethyl-3-methyl-pyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-am inoethyl)am ino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(8-hydroxyethyl)amino-1-methylpyrazole, and the addition salts thereof. 4,5-Diamino-1-(8-methoxyethyl)pyrazole may also be used.

Pyrazole derivatives that may also be mentioned include diamino-N,N-dihydropyrazolopyrazolones such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-on- e, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol- -3-one, 2,3-diamino-6-hydroxy-6,7-dihydro-1H,5H-pyrazolo[1,2-a]pyrazol-1-o- ne. 2,3-Diamino-6,7-dihydro-1H,5H-pyrazolo[1,2-a]pyrazol-1-one and/or a salt thereof will preferably be used. 4,5-Diamino-1-(β-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

Composition according to the present disclosure may optionally comprise one or more couplers advantageously chosen from those conventionally used in the dyeing or coloring of keratin fibers.

Among these couplers, mention may be made especially of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5—N—(β-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methyl-benzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diamino-phenoxy)propane, 3-ureidoaniline, 3-ureido-1-dimethylamino-benzene, sesamol, 1-β-hydroxyethylamino-3,4-methylene-dioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylene-dioxybenzene, 2,6-bis(J-hydroxyethylamino)toluene, 6-hydroxy-indoline, 2,6-dihydroxy-4-methylpyridine, 1-H-3-methylpyrazol-5-one, 1-phenyl-3-methylpyrazol-5-one, 2,6-dimethyl-pyrazolo[1,5b]-1,2,4-triazole, 2,6-dimethyl[3,2-c]-1,2,4-triazole and 6-methylpyrazolo[1,5-a]benzimidazole, the addition salts thereof with an acid, and mixtures thereof.

In general, the addition salts of the oxidation bases and couplers that may be used in the context of the disclosure are especially selected from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the composition, and preferably from 0.005% to 5% by weight relative to the total weight of the compositions of the present disclosure.

The coupler(s), if they are present, each advantageously represent from 0.001% to 10% by weight relative to the total weight of the composition, and preferably from 0.005% to 5% by weight relative to the total weight of the compositions of the present disclosure.

Compositions according to the disclosure may optionally comprise b) one or more synthetic or natural direct dyes, chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Many direct dyes are cationic direct dyes. Mention may be made of the hydrazono cationic dyes of formulas (Va) and (V′a), the azo cationic dyes (VIa) and (VI′a) and the diazo cationic dyes (VIIa) below:

| | |
|---|---|
| Het⁺-C(R^{a})═N—N(R^{b})—Ar, An⁻ | (Va) |
| Het⁺-N(R^{a})—N═C(R^{b})—Ar, An⁻ | (V′a) |
| Het⁺-N═N—Ar, An⁻ | (VIa) |
| Ar⁺—N═N—Ar″, An⁻ | (VI′a) and |
| Het⁺-N═N—Ar′—N═N—Ar, An⁻ | (VIIa) |

in which formulas (Va), (V′a), (VIa), (VI'a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R_{b} with a substituent of Ar and/or R^{a} with R_{b} form, together with the atoms that bear them, a (hetero)cycloalkyl;
particularly, R^{a} and R_{b} represent a hydrogen atom or a group (C₁-C₄)alkyl, which is optionally substituted with a hydroxyl group;
An⁻ represents an anionic counter-ion such as mesylate or halide. In particular, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954, which are incorporated herein by reference in their entirety.

In some cases, the cationic part is derived from the following derivatives:

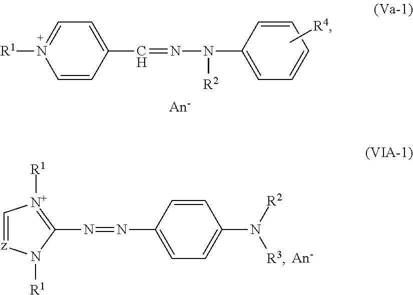

wherein in formulae (Va-1) and (VIa-1):
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH;
An⁻ represents an anionic counter-ion such as mesylate or halide.

Particularly, the dye of formulae (Va-1) and (VIa-1) is chosen from Basic Red 51, Basic Yellow 87 and Basic Orange 31 or derivatives thereof:

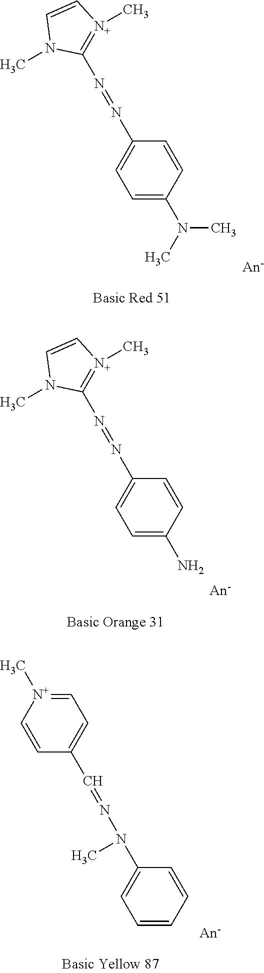

Among the natural direct dyes that may be used according to the disclosure, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the direct dye(s) more particularly represent from 0.001% to 10% by weight and preferably from 0.005% to 5% by weight of the total weight of the compositions of the present disclosure.

### Additional Components

### Cationic Conditioning Compounds

The cationic conditioning agent employed in the compositions of the present invention can be a monoalkyl quaternary amine, such as stearyltrimonium chloride, soyatrimonium chloride or coco-ethyldimonium ethosulfate. Other suitable cationic conditioning agents include, but are not limited to, behentrimonium chloride, dialkyl quaternary amines, such as dicetyldimonium chloride, dicocodimethyl ammonium chloride or distearyldimethyl ammonium chloride; and polyquaternium compounds, such as Polyquaternium-6, Polyquaternium-22 or Polyquaternium-5.

For example, cationic conditioning agents may be chosen from polyquaterium-10 (also called quaternized polyhydroxyethyl cellulose), cetrimonium chloride (also called cetyl trimethyl ammonium chloride, CTAC), behentrimonium chloride (also known as docosyl trimethyl ammonium chloride), behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, am inopropyl dimethicone, Quaterium-8, and dilinoleamidopropyl dimethylamine dimethicone PEG-7 phosphate.

In some instances, the cationic conditioning agents are cationic conditioning polymers. Examples of cationic conditioning polymers that can be used include, without limitation, cationic cellulose, cationic proteins, and cationic polymers. The cationic polymers can have a vinyl group backbone of amino and/or quaternary ammonium monomers. Cationic amino and quaternary ammonium monomers include, without limitation, dialkylamino alkylmethacrylate, monoalkylaminoalkyl acrylate, monoalkylaminoalkyl methacrylate, trialkyl methacryoloxyalkyl ammonium salt, trialkyl acryloxyalkyl ammonium salts, diallyl quaternary ammonium salts, vinyl compounds substituted with dialkyl aminoalkyl acrylate, and vinyl quaternary ammonium monomers having cyclic cationic nitrogen containing rings such as pyridinium, imidazolium, or quaternized pyrrolidine. Other examples of cationic conditioning polymers that can be used include, without limitation, hydroxypropyltrimonium honey, cocodimonium silk amino acids, cocodimonium hydroxypropyl hydrolyzed wheat or silk protein, polyquaternium-5, polyquaternium-11, polyquaternium-2, polyquaternium-4, polyquaternium-6, polyquaternium-7, polyquaternium-14, polyquaternium-16, polyquaternium-22, polyquaternium-10, and guar hydroxypropyltrimonium chloride.

In some cases quaternized polymeric cationic conditioning agents are particularly useful. Particularly preferred are quaternary nitrogen polymers prepared by the polymerization of a dialkyldiallylammonium salt or copolymer thereof in which the alkyl group contains 1 to about 18 carbon atoms, and more preferably where the alkyl group is methyl or ethyl. Details concerning the preparation of these polymers can be found in U.S. Pat. Nos. 3,288,770, 3,412,019 and 4,772,462, incorporated herein by reference. For example, cationic homopolymers and copolymers of polydiallyldimethylammonium chloride are available in aqueous compositions sold under the trademark MERQUAT by the Calgon Corporation, subsidiary of Merck & Co., Pittsburgh, Pa. The homopolymer, which is named Polyquaternium-6 in the CTFA Cosmetic Ingredient Dictionary, 3rd Ed., published in 1982 by the Cosmetic Toiletry and Fragrance Association, Inc. (hereafter CTFA Dictionary and CTFA name), is sold under the trademark MERQUAT-100, and is described as having a weight average molecular weight of approximately 100,000. A copolymer reaction product of dimethyldiallylammonium chloride with acrylamide monomers is named Polyquaternium-7 in the CTFA Dictionary, is described as having a weight average molecular weight of approximately 500,000 and is sold under the trademark MERQUAT-550. Another copolymer reaction product of dimethyldiallylammonium chloride with acrylic acids having a weight average molecular weight from about 50,000 to about 10,000,000 has the CTFA name Polyquaternium-22 and is sold under the trademark MERQUAT-280. Polyquaternium-6 is particularly preferred.

Other polymeric conditioners include cationic copolymers of methylvinylimidazolium chloride and vinyl pyrrolidone, sold commercially by BASF Aktiengesellschaft, West Germany under the trademark LUVIQUAT at three comonomer ratios, namely at ratios of 95/5, 50/50 and 30/70 methylvinylimidazolium chloride to polyvinylpyrrolidone. These copolymers at all three comonomer ratios have the CTFA name Polyquaternium 16. Polymeric conditioners also include cationic cellulosic polymers of hydroxyethyl cellulose reacted with epichlorohydrin and quaternized with trimethylamine, sold under the trademark POLYMER JR in various viscosity grades and molecular sizes by Union Carbide Corporation, Danbury, Conn. These series of polymers are named Polyquaternium 10 in the CTFA Dictionary. Also useful are quaternized copolymers of hydroxyethylcellulose and dimethyldimethylammonium chloride, having the CTFA name Polyquaternium-4, sold in varying molecular weights under the trademark CELQUAT by National Starch and Chemical Corporation, Bridgewater, N.J.

Smaller molecule cationic non-polymeric conditioning agents can also be utilized herein. Exemplary small-molecule conditioning agents can include monofunctional or difunctional quaternary ammonium compounds, such as stearyldimethylbenzylammonium chloride, dimethyldi-(hydrogenated tallow)ammonium chloride, and the like. Non-polymeric conditioning agents can also include the quaternary ammonium salts of gluconamide derivatives, such as gamma-gluconam idopropyldimethyl-2-hydroxyethyl-ammonium chloride and minkamidopropyldimethyl-2-hydroxyethylammonium chloride identified respectively by the CTFA names Quaternium 22 and Quaternium 26. Details for the preparation of these materials are found in U.S. Pat. Nos. 3,766,267 and 4,012,398, respectively, and the materials are sold under the trademark CERAPHYL by Van Dyk & Co., Belleville, N.J. Also useful are bis-quaternary ammonium compounds which are dimers, such as 2-hydroxy propylene-bis-1,3-(dimethylstearyl ammonium chloride, designated the CTFA name, Hydroxypropyl Bisstearyldimonium chloride. The preparation of these and other bis-quat materials is described in U.S. Pat. No. 4,734,277, and such materials are sold under the trademark JORDAQUAT DIMER by Jordan Chemical Company, Folcroft, Pa.

Exemplary unquaternized polymers having tertiary amino nitrogen groups that become quaternized when protonated can include water-soluble proteinaceous quaternary ammonium compounds. Cocodimonium hydrolyzed animal protein, for example, is the CTFA name for a chemically-modified quaternary ammonium derivative of hydrolyzed collagen protein having from about 12 to about 18 carbons in at least one aliphatic alkyl group, a weight average molecular weight from about 2500 to about 12,000, and an isoionic point in a range from about 9.5 to about 11.5. This material and structurally related materials are sold under the trademarks CROQUAT and CROTEIN by Croda, Inc., New York, N.Y.

The total amount of the one or more conditioning agents, if present, may vary. In some cases, the total amount of the one or more conditioning agents is from about 0.1 to about 25 wt. %, about 0.1 to about 20 wt. %, about 0.1 to about 15 wt. %, 0.1 to about 10 wt. %, 0.1 to about 5 wt. %, about 1 to about 25 wt. %, about 1 to about 20 wt. %, about 1 to about 15 wt. %, about 1 to about 10 wt. %, or about 1 to about 5 wt. %, based on the total weight of the composition.

### Thickening Agents

The compositions may contain one or more thickeners or viscosity modifying agents. Classes of such agents include, but are not limited to, viscous liquids, such as polyethylene glycol, semisynthetic polymers, such as semisynthetic cellulose derivatives, synthetic polymers, such as carbomers, poloxamers, and polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, and combinations thereof.

The total amount of the one or more thickening agents may vary, but in some cases is about 0.1 to about 15 wt. %, about 0.1 to about 10 wt. %, about 0.1 to about 8 wt. %, about 0.1 to about 6 wt. %, about 0.1 to about 5 wt. %, about 0.5 to about 10 wt. %, about 0.5 to about 8 wt. %, about 0.5 to about wt. %, about 0.5 to about 5 wt. %, about 1 to about 10 wt. %, about 1 to about 8 wt. %, about 1 to about 6 wt. %, or about 1 to about 5 wt. %, based on the total weight of the composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the composition. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol).

The total amount of the one or more preservatives, when present, may vary. In some cases, the total amount of the one or more preservatives is about 0.01 to about 5 wt. %, about 0.01 to about 4 wt. %, about 0.15 to about 1 wt. %, or about 1 to about 3 wt. %, based on the total weight of the composition.

Suitable components, such as those listed in the instant disclosure (including those listed above), may be included or excluded from the hair care formulations depending on the specific combination of other components, the form of the compositions, and/or the use of the formulation (e.g., hair spray, cream, conditioner, etc.).

### Example Types of Active Compositions

### Bleach Compositions

When hair lightening or color-altering uses separate bleach and developer compositions, the bleach composition may comprise at least one oxidizing agent chosen from persulfates, perborates, percarbonates, peracids, bromates, their salts, and mixtures thereof. In some instances, the at least one oxidizing agent is chosen from alkali metal salts of perborates, percarbonates, bromates, and persulfates, such as, for example, ammonium, sodium, and potassium salts. The bleach composition may also optionally comprise a cosmetically acceptable carrier.

The at least one oxidizing agent of the bleach compositions of the disclosure is utilized in an amount sufficient to lighten or “bleach” hair. By way of example only, the at least one oxidizing agent of the bleach composition may be present in an amount ranging from about 10% by weight to about 100% by weight, such as from about 20% to about 90% by weight, from about 30% to about 80% by weight, or from about 40% to about 75% by weight, based on the total weight of the bleach composition. In further embodiments, the at least one oxidizing agent of the bleach composition may be present in an amount ranging from about 5% to about 50%, such as about 10% to about 45%, or about 15% to about 40%. In some cases, the at least one oxidizing agent of the bleach composition may be present in an amount of at least 40% by weight, based on the total weight of the bleach composition.

The bleach composition may be in any form, such as, for example, in the form of a powder, gel, liquid, foam, lotion, cream, mousse, and emulsion.

In some cases, the bleach composition may be anhydrous. Optionally, water may be added as an activator, by mixing it with the bleach composition.

The bleach composition of the present dislosure may also contain acid and alkali pH adjusters, which are well known in the art in the cosmetic treatment of keratin fibers, such as hair. Such pH adjusters include, but are not limited to, sodium metasilicate, silicate compounds, citric acid, ascorbic acid, and carbonate compounds.

The pH adjusters may, in various embodiments, be present in the bleach composition in an amount effective to provide the color-altering composition with a pH ranging from about 1 to about 7 when the bleach composition is combined with the developer composition. By way of example, the amount of pH adjuster may be present, in various embodiments, in an amount of at least about 0.01%, such as at least about 0.1%, at least about 0.2%, or at least about 0.5%.

In some cases, the bleach composition is acidic, with the pH ranging from about 1 to about 7; and in some cases, the bleach composition has a pH higher than about 7. When the bleach composition is in powder form, the pH may be measured in a 1% solution in water.

Colorants may also optionally be present in the bleach compositions. The colorants useful according to various embodiments of the disclosure are those colorants that are stable in the bleach composition, and can impart additional toning and coloring to hair. Exemplary hair colorants include, but are not limited to, pigments, liposoluble dyes, direct dyes, nacreous pigments, pearling agents, leuco dyes, optical lightening colorants, natural colorants and optically-variable pigments.

### Developer Compositions

Developer compositions often include peroxide, such as hydrogen peroxide. The developer composition may also optionally comprise a cosmetically acceptable carrier. The hydrogen peroxide may be present in an amount of at least about 1% by weight, based on the total weight of the developer composition. In some cases, hydrogen peroxide is present in an amount ranging from about 0.1% to about 80% by weight, such as from about 1.0% to about 75% by weight, or from about 2% to about 10% by weight, based on the total weight of the developer composition. Furthermore, the hydrogen peroxide may be present in the developer composition in an amount ranging from about 2% to about 25%, such as about 4% to about 20%, about 6% to about 15%, or about 7% to about 10%.

The cosmetically acceptable carrier of the developer composition may, for example, be present in an amount ranging from about 0.5% to about 99% by weight, such as from about 5% to about 95% by weight, relative to the total weight of the developer composition.

The pH of the developer composition can range from about 1 to about 5, such as from about 2 to about 4, and it may be adjusted to the desired value using pH adjusters that are well known in the art in the cosmetic treatment of keratin fibers, including, for example, those described herein.

The developer composition may be in the form of a powder, gel, liquid, foam, lotion, cream, mousse, and emulsion.

Optionally, water may be added as an activator, by mixing it with the developer composition.

The developer composition may, in various cases, comprise additional components such as, for example, at least one auxiliary ingredient chosen from rheology-modifying agents, chelants, fatty substances, ceramides, alkoxyaminosilicones, and silanes, and any other component known in the art to be useful in a developer composition.

In some instances, the bleach composition and developer composition may be combined to form the lightening composition or color-altering composition in a ratio of bleach composition to developer composition ranging from about 1:1 to about 1:5, such as from about 1:1 to about 1:2, or about 1:2 to about 1:4.

### Shape-Altering Compositions

Compositions for altering the shape of the hair comprise hair shaping agents for example, agents for straightening, relaxing, and/or shaping the hair. By way of example, hair shaping agents may optionally be chosen from inorganic hydroxides or organic hydroxides, for example sodium hydroxide, potassium hydroxide, lithium hydroxide, calcium hydroxide, or guanidine hydroxide, or may be chosen from organic amines and other non-hydroxide compounds. In some cases, the hair relaxing agents may be chosen from thiol compounds such as cysteine, cysteamine, N-substituted cysteamines, alkyl substituted mercaptoacetamides, dimercaptoadipic acid, thioglycerol, thiolactic acid, thioglycolic acid or its salts, (e.g., a thioglycolate), monothioglycolic acid esters such as diol esters of thioglycolic acid, glyceryl monothioglycolate, thiocholine or its salts, amino thiols, and thiols attached to low molecular weight polymers, sulfites such as sodium hyposulfite, and bisulfites such as ammonium or sodium bisulfite.

The hair shaping composition may also comprise a cosmetically acceptable carrier. The cosmetically acceptable carrier may, for example, be present in the shape-altering composition in an amount ranging from about 1% to about 40% by weight, such as from about 5% to about 35% by weight, or about 10% to about 30% by weight of the shape-altering composition.

In some cases, the hair shaping composition comprises or is used in conjunction with at least one neutralizer (for example, in a neutralizing composition), for example an oxidizing agent. Exemplary useful oxidizing agents include peroxides, bromates, and perborates, e.g., hydrogen peroxide, potassium bromate, sodium bromate and sodium perborate.

The shape-altering composition may be left on the hair for a period of time sufficient to achieve the desired alteration in hair shape. For example, the shape-altering composition may be left on the hair for up to one hour, such as from about 3 minutes to about 45 minutes, from about 5 minutes to about 30 minutes, or from about 10 minutes to about 20 minutes. In further embodiments, the shape-altering composition may be left on the hair for a period up to about 30 minutes, such as, for example, from about 1 to about 30 minutes, about 1 to about 10 minutes, or about 1 to about 5 minutes. One skilled in the art will, by considering various factors such as starting hair shape and desired hair shape, be able to determine an appropriate amount of time to leave the shape-altering composition on the hair in order to achieve the desired alternation in hair shape.

If desired, the shape-altering composition may, optionally, be shampooed and/or rinsed off the hair.

### Forms

The compositions described herein may be in any suitable physical form.

Suitable forms include, but are not limited to low to moderate viscosity liquids, lotions, milks, mousses, sprays, gels, creams, conditioners, and the like.

i. Spray

The compositions described herein for treating hair may be in the form of a spray. The spray typically includes a cosmetically acceptable carrier. In some embodiments, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant.

The hair spray formulations may be dispensed from containers that include aerosol dispensers or pump spray dispensers. Such dispensers are known in the art and are commercially available from a variety of manufacturers.

When the hair spray formulation is dispensed from a pressurized aerosol container, a propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

The amount of propellant may range from about 10% to about 60% by weight of the formulation. The propellant may be separated from the hair repair formulation as in a two compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the hair strengthening formulation to the hair.

ii. Conditioners

The compositions disclosed herein may be in the form of a conditioner. The conditioner may include one or more conditioning agents, such as cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example with cationic or neutral groups. These polymers may have a stabilizing or strengthening action on the compositions, and/or a conditioning action (deposition on the surface of the skin or the hair).

iii. Creams

The compositions disclosed herein for may be in the form of a cream.

Implementation of the present disclosure is provided by way of the following examples. The examples serve to illustrate the technology without being limiting in nature.

### EXAMPLE 1

### Formulations

| | | | | | Control | Control |
|---|---|---|---|---|---|---|
| Ingredients | 1 | 2 | 3 | 4 | 1 | 2 |
| Maleic Acid | 10.8 | — | 10.8 | 10.8 | 10.8 | |
| Malonic Acid | — | 9.6 | | | | 9.6 |
| Mono- | 5.4 | 4.0 | 5.4 | 4.0 | 5.4 | 4.0 |
| ethanoloamine | | | | | | |
| PVM/MA | 1.5 | 1.5 | | | — | — |
| Copolymer¹ | | | | | | |
| PVM/MA | | | 1.5 | | — | — |
| Copolymer | | | | | | |
| (low MW)² | | | | | | |
| PVM/MA | | | | 1.5 | — | — |
| Copolymer | | | | | | |
| (high MW)³ | | | | | | |
| Water | QS 100 | QS 100 | QS 100 | QS 100 | QS 100 | QS 100 |

¹Present in the raw material known by the tradename STYLEZE XT3 and commercially available from Ashland. INCI Name: Aqua (and) Polyimide-1 (and) PVM/MA Copolymer (and) Caprylyl Glycol.
²Present in the raw material known by the tradename GANTREZ AN-139 and commercially available from Ashland. GANTREZ AN-139 is a copolymer of methyl vinyl ether and maleic anhydride.
³Present in the raw material known by the tradename GANTREZ 169 BF and commercially available from Ashland. GANTREZ 169 BF is a copolymer of methyl vinyl ether and maleic anhydride.

### EXAMPLE 2

### Cosmetic Properties

15 grams of a bleach powder was mixed with 15 grams of a beach developer composition, and 4 grams of Formulation 1, 2, 3, or 4 of Example 1 was added. Each of the four mixtures was separately applied to hair swatches for 35 minutes. After 35 minutes, the swatches were rinsed with water, shampooed once, and blown dry. The components of the bleach composition and the developer composition are provided below.

### Bleach Composition

| Ingredient | wt. % |
|---|---|
| Magnesium Carbonate Hydroxide | 9 |
| Sodium Silicate | 17 |
| Disodium EDTA | 1 |
| Sodium Metasilicate | 3 |
| Surfactants | 11 |
| Potassium Persulfate | 50 |
| Ammonium Persulfate | 5 |
| Additives and Optional Ingredients | 4 |

### Developer Compositions

| Ingredient | wt. % | wt. % |
|---|---|---|
| Sodium Stannate | 0.04 | 0.04 |
| Surfactants | 3.7 | 3.7 |
| Pentasodium Pentetate | 0.06 | 0.06 |
| Glycerin | 0.5 | 0.5 |
| Hydrogen Peroxide | 9 | 12 |
| Tetrasodium Pyrophosphate | 0.02 | 0.02 |
| Water | QS 100 | QS 100 |

The treated hair swatches were evaluated by three independent experts for sensorial characteristics (combability, smoothness, and discipline). Each individual expert ranked the hair swatches according to a four point scale, where “1” represents the best result and “4” represents the worst result. All three experts arrived at the same conclusions, which are shown below.

| | Combability | Smoothness | Discipline |
|---|---|---|---|
| No Additive¹ | 4 | 4 | 4 |
| Control 1 | 2 | 2 | 2 |
| Control 2 | 2 | 2 | 2 |
| Formulation 1 | 1 | 1 | 1 |
| Formulation 2 | 1 | 1 | 1 |
| Formulation 3 | 1 | 1 | 1 |
| Formulation 4 | 1 | 1 | 1 |

¹Hair bleached using only the combination of bleach powder and bleach developer (1:1 ratio) (no maleic acid, no monoethanolamine, no maleic acid polymer).

Hair bleached with no additive provided the worst cosmetic properties, as it was most difficult to comb, it was rough, and exhibited the least amount of discipline. The addition of a di-carboxylic acid (maleic acid or malonic acid) with an amine (monoethanol amine), as with Control 1 and 2, provided some improvement to cosmetic properties. However, the combination of a polymeric acid compound and/or a polymeric acid anhydride compound with the di-carboxylic acid and amine exhibited the very best results, as shown by Formulations 1-4.

### EXAMPLE 3

### Restructuring Properties

15 grams of bleach powder was mixed with 15 grams of beach developer, and 4 grams of Formulation 1 of Example 1 was added. Separately, 15 grams of the bleach powder was mixed with 15 grams of the beach developer, but no additional components were added (no additives). The bleach composition and the developer were the same as in Example 2 above.

Each mixture was separately applied to hair swatches for a minimum of 50 minutes, or until the treatment with Formulation 1 and the treatment without additives had matching lifts as measured by L*a*b*. The swatches were rinsed with water, shampooed once, and blown dry. The swatches were tested with a Miniature Tensile Tester (MTT) and subjected to Amino Acid Analysis (AAA). The results are presented in the tables below.

### Miniature Tensile Tester (MTT)

| Treatment | n (fibers) | Elastic Modulus | Break Stress |
|---|---|---|---|
| No Additive | 45 | 687.2 (±139.3) | 92.2 (±17.5) |
| Formula 1 | 49 | 922.1 (±226.1) | 114.9 (±15.1) |

### Amino Acid Analysis

| | % Difference from “No |
|---|---|
| | Additive” (Cysteic Acid) |
| Formula 1 | 5.9 |

As shown by the data above, both Elastic Modulus and Break Stress are higher when hair is treated with an inventive formulation than when hair is treated with a formulation without additives. Additionally, less cysteic acid is formed when the inventive formulation is used indicating much less damage to the hair. These results demonstrate that hair treated with the inventive formulation (mixture with Formulation 1) surprisingly exhibited significantly less damage after chemical treatment and possessed increased strength in comparison to hair treated without the inventive formulation.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular.

The expression “one or more” means “at least one” and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5% of the indicated number.

All percentages, parts and ratios herein are based upon the total weight of the compositions of the present invention, unless otherwise indicated.

“Keratinous substrates” as used herein, includes, but is not limited to keratin fibers such as hair and/or scalp on the human head.

“Conditioning” as used herein means imparting to one or more hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of keratinous substrates such as hair. The term “treat” (and its grammatical variations) as used herein also refers to contacting keratinous substrates such as hair with the compositions of the present disclosure.

A “rinse-off” product refers to a composition such as a hair care composition that is rinsed and/or washed with water either after or during the application of the composition onto the keratinous substrate, and before drying and/or styling said keratinous substrate. At least a portion of the composition is removed from the keratinous substrate during the rinsing and/or washing.

The term “stable” as used herein means that the composition does not exhibit phase separation and/or crystallization.

“Volatile”, as used herein, means having a flash point of less than about 100° C.

“Non-volatile”, as used herein, means having a flash point of greater than about 100° C.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All ranges and values disclosed herein are inclusive and combinable. For examples, any value or point described herein that falls within a range described herein can serve as a minimum or maximum value to derive a sub-range, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair restructuring composition comprising:
(a) one or more non-polymeric, mono-, di-, and/or tri-carboxylic acids;
(b) one or more amines; and
(c) one or more polymeric acid compounds and/or polymeric acid anhydride compounds containing recurring units derived from maleic acid and/or maleic anhydride.

2. The hair restructuring composition of claim 1 comprising one or more di-carboxylic acids.

3. The hair restructuring composition of claim 2 comprising one or more di-carboxylic acids selected from the group consisting of oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, and mixtures thereof.

4. The hair restructuring composition of claim 2 comprising maleic acid and/or malonic acid.

5. The hair restructuring composition of claim 1 comprising:
(a) about 1 to about 50 wt. % of the one or more non-polymeric, mono-, di-, and/or tri-carboxylic acids.

6. The hair restructuring composition of claim 1 comprising one or more amines selected from the group consisting of monoamines, diamines, polyamines, and mixtures thereof.

7. The hair restructuring composition of claim 6 comprising one or more monoamines selected from the group consisting of monoethanolamine, propanolamine, isopropanolamine, triethanolamine, butanolamine, isobutanolamine, methylethanolamine, butylethanolamine, and mixtures thereof.

8. The hair restructuring composition of claim 1 comprising:
(b) about 0.5 to about 50 wt. % of the one or more amines.

9. (canceled)

10. (canceled)

11. The hair restructuring composition of claim 1 comprising:
(c) about 0.01 to about 25 wt. % of the one or more one or more polymeric acid compounds and/or polymeric acid anhydride compounds.

12. A composition for chemically treating hair comprising:
(a) one or more non-polymeric, mono-, di-, and/or tri-carboxylic acids;
(b) one or more amines; and
(c) one or more polymeric acid compounds and/or polymeric acid anhydride compounds containing recurring units derived from maleic acid and/or maleic anhydride; and
(d) one or more active agents.

13. The composition of claim 12 comprising one or more active agents selected from the group consisting of oxidizing agents, reducing agents, non-reducing agents for shaping hair, dyeing agents, and mixtures thereof.

14. The composition of claim 13 that is a hair lightening composition comprising one or more oxidizing agents selected from the group consisting of one or more peroxides, persulfates, perborates, percarbonates, and mixtures thereof.

15. The composition of claim 13 that is selected from the group consisting of:
(i) a hair perming or straightening composition comprising one or more reducing agents selected from the group consisting of cysteine or a derivative of cysteine, cysteamine or a derivative of cysteamine, thiolactic acid or an ester of thiolactic acid, thioglycolic acid or an ester of thioglycolic acid, thioglycerol, and mixtures thereof; and
(iii) a hair straightening or relaxing composition comprising one or more non-reducing agents for shaping hair selected from the group consisting of hydroxide compounds, non-hydroxide compounds, and mixtures thereof.

16. The composition of claim 13 that is a hair-coloring composition comprising one or more dyeing agents selected from the group consisting of direct dyes, oxidative dyes, direct action dyes, natural dyes, metallic dyes, reactive dyes, and mixtures thereof.

17. A method for restructuring hair comprising applying a hair restructuring composition of claim 1 to the hair, wherein the method is carried out at a temperature between 10° C. and 50° C.

18. A method for chemically treating hair comprising:
i. applying to the hair a composition for chemically treating the hair of claim 12:
ii. allowing the composition to remain on the hair for about 1 to about 45 minutes; and
iii. rinsing the composition from hair;
wherein the method is carried out at a temperature between 10° C. and 50° C.

19. A kit comprising:
i. a restructuring composition of claim 1;
ii. a composition comprising one or more active agents for chemically treating hair; and
iii. optionally, a composition comprising one or more hair conditioning agents.

20. A kit comprising:
i. a composition for chemically treating hair of claim 12; and
ii. a composition comprising one or more second active agents that is different than the one or more active agents in (i); and
iii. optionally, a composition comprising one or more hair conditioning agents.

21. A hair restructuring composition comprising:
(a) about 1 to about 50 wt. % of maleic acid and/or malonic acid;
(b) about 0.5 to about 50 wt. % of monoethanolamine; and
(c) about 0.01 to about 25 wt. % of one or more polymers containing recurring units derived from maleic acid and/or maleic anhydride.
